(19) United States
(12) Patent Application Publication
(10) Pub. No.: US 2024/0200074 A1
(43) Pub. Date: Jun. 20, 2024

(54) METHOD AND COMPOSITIONS FOR PREVENTING TUMOR DEVELOPMENT AND METASTASIS

(71) Applicant: The Administrators of the Tulane Educational Fund, New Orleans, LA (US)

(72) Inventors: Eckhard ALT, Houston, TX (US); Reza IZADPANAH, New Orleans, LA (US)

(21) Appl. No.: 18/555,477

(22) PCT Filed: Apr. 13, 2022
(86) PCT No.: PCT/US22/24700

§ 371 Date: Oct. 13, 2023

Related U.S. Application Data
(60) Provisional application No. 63/174,527, filed on Apr. 13, 2021.

(51) Int. Cl. C12N 15/113; A61K 45/06; A61P 35/04

(52) U.S. Cl. CPC C12N 15/113; A61K 45/06; A61P 35/04; C12N 2310/11; C12N 2310/14; C12N 2310/141; C12N 2310/531

(57) ABSTRACT

A method for preventing and treating micrometastasis in a patient with cancer, especially breast cancer or glioblastoma tumors, by silencing TRAF3IP2 before during or in connection with treatment as described herein.

### CROSS-REFERENCES TO RELATED APPLICATIONS

This application claims priority to U.S. App. No. 63/174,527, filed on Apr. 13, 2021, which is incorporated herein in its entirety for all purposes.

### STATEMENT REGARDING FEDERALLY SPONSORED RESEARCH

### Field of the Invention

The present invention relates to novel methods to prevent tumor metastasis and suppress tumor growth, especially of solid tumors, by interfering with tumor communication and its environment and by impacting the formation and development of the tumor microenvironment.

### BACKGROUND OF THE INVENTION

Cancer mortality and the multitude of unsuccessful treatments primarily originate not from the growth of the primary tumor but from metastases. In many cases the metastatic spread is induced by manipulation on a tumor, predominantly by an operation or diagnostic procedure. Tumor development occurs following the accumulation of genetic and epigenetic alterations in tumor cells. It has been demonstrated that tumor growth is strongly influenced by non-malignant cells that together with the tumor cells form the tumor microenvironment.

Micrometastasis refers to a small collection of cancer cells that have been shed from the original tumor and spread to another part of the body through the blood or lymph nodes. The cancer cells group together and form a second tumor that is not yet detectable with imaging test, such as mammogram or MRI, but can only be seen under a microscope. The clusters of cancer calls are between 0.2 mm to 2.0 mm in diameter.

It has been reported that the metastatic possess consists of three steps. First tumor cells have to enter the systemic circulation, which is difficult for them without manipulation. Then, circulating cells need to leave the circulatory system and to cross the endothelial barrier. Lastly, the cancer cells need to migrate through tissue in order to encraft and to form tiny micrometastases. While very few cells are at all capable to form a metastasis, primarily and most likely only the so-called tumor stem cells are capable to initiate the metastatic process.

Current mortality statistics and tumor statistic evidence that the metastatic occurrence often results in a time-dependent manner related to an intervention such as an operation, intervention or biopsy. For example, from operative resections of colon cancer—that initially at time of the operation had no evident metastasis—it is known, that in 30% of these patients a liver metastasis 3 to 6 month after an operation occurs. It has been proposed that surgery may cause or facilitate metastasis by allowing the cancer stem cells to enter the circulation. Baum et al., (2005); Retsky et al., (2005). Further, it is also proposed that chemotherapy may activate cancer stem cells. Lathia (2015). That suggests that the metastatic process is triggered or enhanced by the operation.

Additionally, TRAF3 Interacting Protein 2 (TRAF3IP2), an upstream regulator of both transcription factors NF-κB and AP-1-responsive genes, has been proposed to be a target of reducing tumor growth by inhabiting NF-κB and pro-inflammatory/pro-tumorigenic mediators in glioblastoma. Alt et al. (2018).

Although the importance of microenvironmental alterations in tumor development is recognized, the molecular mechanisms underlying these changes are only now beginning to be understood. Detailed molecular characterization of various cell types from normal breast tissue, ductal carcinoma, and invasive breast tumors has revealed that gene expression changes occur in all cell types during breast tumor progression.

The present invention provides novel compositions and methods to affect the interactions between a tumor and its microenvironment to prevent, reverse, and/or reduce metastasis.

### BRIEF SUMMARY OF THE DISCLOSURE

The present disclosure provides a novel method for preventing micrometastasis caused by invasive procedures. The method includes administering a pharmaceutical composition to a patient undergoing an invasive procedure in a cancer before, during or in connection to the intervention, in which TRAF3IP2 is overexpressed at least 5 fold compared to a non-cancerous somatic cell of the patient. The pharmaceutical composition comprises at least one silencing sequence for TRAF3IP2 in a pharmaceutically acceptable carrier in an amount effective for the prevention of micrometastasis. In some embodiments, the silencing sequence reduces the expression of the TRAF3IP2 gene by at least 5-fold as compared to without the silencing sequence for TRAF3IP2. This disclosure describes the causal relationship between TRAF3IP2 and metastasis, as well as the reduction in metastasis and neoangiogenesis by silencing TRAF3IP2 in the tumor tissue.

In another aspect of this disclosure, a method of treating cancer is described, wherein the cancer overexpresses at least 5-fold of TRAF3IP2 as compared to a control. The method comprises administering a pharmaceutical composition to a subject having the cancer, followed by performing at least one of surgery, chemotherapy, radiation therapy, immunotherapy or targeted therapy.

For tumors, especially solid tumors, including breast cancers or glioblastomas, by interfering with tumor communication with the tumor environment and/or by regressing formation of the microenvironment, thereby preventing or reversing tumor metastasis and suppressing tumor growth

Silencers can be delivered to a tumor in a number of ways, including at least:

1) Delivering silencing RNA by injecting an expression vector encoding the silencer to the tumor site, e.g., directly into a tumor site under visual, ultrasound, fluoroscopy, CT or MRI guidance or other imaging modalities, or indirectly through blood vessels or ducts that lead to the tumor.

2) Use of silencing RNA delivered by tumor targeting cells, such as migratory stem cells, e.g., MSCs, or any type of cells that due to their nature preferably migrate and engraft to the tumor site. Such cells would contain therein either an expression vector or a genomic copy of the sequence encoding the silencer.

3) Delivering encapsulated or otherwise protected silencing RNA to the tumor site. The silencing RNA is for example encapsulated into microspheres (i.e. exosomes) or micelles, liposomes and the like. The microspheres will be delivered by direct or indirect injection to the tumor site either through a transcutaneous injection or through a vessel or duct supplying the tumor site. Preferably, such RNAs will be RNAse resistant, and if so, naked RNA may be used.

4) Silencing RNA linked to a specific tumor directed antibody or protamine coupled construct to increase the tumor specific concentration and to enhance the local effect of the silencing RNA within the tumor site.

5) Achieving a selective effect targeting the tumor cells and virtually avoiding an effect on non-tumor cells by i) increasing the local concentration within the tumor by selective delivery means as described above, ii) by the fact that the respective genes of TRAF3IP2 and of Rab27a are significantly up to 250 times (respectively) upregulated in tumor, especially in tumor stem cells, compared to normal stem cells, and iii) the silencer is released in a (transactivator)-inducible manner (such as IL1B), thus expression is activated mainly in the tumor.

6) Combinations and variations of the above.

After the silencing RNA has been delivered to the tumor site, surgery and/or chemotherapy can be performed. This is because silencing TRAF3IP2 in tumor cells confines cytokine expression and ultimately limits the development of the tumor microenvironment that could facilitate micrometastasis, and eventually slows or prevents tumor growth and restrains tumor metastasis. The tumor cells exhibit significantly higher levels of exocytosis activities compared to non-malignant cells.

For the actual silencer sequence used in this disclosure, an shRNA having the sequence of SEQ ID NO. 1, or an anti-sense oligonucleotide having the sequence of SEQ ID NO. 2 is used. However, any type of silencer for TRAF3IP2 could be used.

Basic design rules for the various types of silencers are available, and once designed the silencers can be tested for efficacy according to the methods discussed herein and in the literature.

For example, a short hairpin silencer (shRNA) generally has about 18-30 nucleotides (nt), preferably 21 nt, comprising a unique sense strand of target mRNA beginning with AA linked to a loop (3-9 nt) linked to an complement of the unique sense strand and finishing with polyT, thus forming a hairpin. An initiating G nt could also be used.

Another type of silencer, is the siRNA of about 18-30 nt, preferably 21 nt, comprising a unique sense strand of the target mRNA beginning with AA and finishing with polyT.

Another type of silencer is the antisense sequence. These can be a unique antisense sequence from the target, or an RNAse resistant 18-30 nt antisense RNA sequence from the target. Effective antisense silencers may also be located in exons, but close to the acceptor splice site (SS).

miRNAs generally work when about 21-23 nt and have complementarity maintained in the first third of the small RNA and target mRNA, but mismatches arise in the remainder of the aligned sequence.

The above rules are guidelines only, however, and there is certainly variability in approaches. Therefore, it is typical to design 4-6 such silencers using the basic rules and then test each for activity, e.g., in an ex vivo system. Therefore, given the validity of the target, silencers can be readily be designed using the target sequence.

There are also non-viral methods of silencer delivery, including e.g, injecting naked DNA/RNA into a tumor, injected protected RNA into tumors, electrotransfection, the use of polymers, liposomes, and the like, to protect the nucleic acids, or to stabilize the silencer through linking it to Protamin.

Lentiviral vectors were used herein to encode the silencer sequences for TRAF3IP2. Although data show that there is specificity for CD45+ cells transduction in vivo when administering lentiviral vectors, MDA-MB231 and SW620 cells are highly transducible with lentiviral vectors. Thus, these vectors were useful for proof of concept studies. However, any suitable expression vector may be used herein, or the gene can be introduced into the genome of a homing cell (e.g., by homologous recombination), such as the MSCs discussed herein.

Common vectors are based on herpes simplex type 1 recombinant vector (HSV-1); adenovirus, adeno-associated viral vector (AAV); alpha virus; vaccinia virus; pox virus; sendai virus; plasmids; retrovirus; ssDNA vectors; and the like. To date, adenovirus, retrovirus and naked plasmid DNA have made up more than half of the vectors tested in clinical trials of various gene therapies.

An IL1B transactivator-inducible system is a preferred promoter for use in our lentiviral vector. The ILIB promoter activates the expression of silencer RNA by binding the endogenous ILIB, which is highly produced by cells within tumor microenvironment. However, this promoter is exemplary only and there are many to choose from, including several antibiotic resistance or drug responsive promoters that can be safely used in humans (e.g., tamoxifen, tetracyclin, ampicillin and the like).

The disclosure provides one or more of the following embodiments, in any combinations(s) thereof:

A method of preventing surgery and inter-
vention related micrometastasis on a
subject having cancer, wherein the cancer
cells have increased TRAF3IP2 expression
comparing to a normal cell, said method
comprising the steps of: a) administering
a pharmaceutical composition into a
subject, and b) performing (i) surgery to
treat the cancer, (ii) diagnostic for the
cancer, or (iii) intervention to treat the
cancer; wherein the pharmaceutical compo-
sition comprises at least one targeting
sequence for TRAF3IP2 in a pharmaceutically
acceptable carrier in an amount effective
for the prevention of micrometastasis,
wherein said silencing sequence reduces
the expression of the TRAF3IP2 gene by at
least 5-fold as compared to without the
silencing sequence for TRAF3IP2.
Any method herein described, wherein the
cancer is breast cancer or glioblastoma
multiforme.
Any method herein described, the targeting
sequence for TRAF3IP2 is selected from the
group consisting of: siRNA, an miRNA, an
shRNA, an antisense RNA, or an antisense
oligonucleotide.
Any method herein described, the composition
comprises an expression vector encoding the
targeting sequence for TRAF3IP2 operably
coupled to an inducible promoter.
Any method herein described, the targeting
sequence for TRAF3IP2 is
CCGGCATGGAACTATCATTACCATTCTCGAGAATGGTAATGAT
AGTTCCATGTTTTTT (SEQ ID NO: 1).
Any method herein described, the targeting
sequence for TRAF3IP2 is an anti-sense
oligonucleotide having the sequence of:
mG*mG*mU*mG*mG*G*C*A*C*A*T*G*C*T*C*mC*mU*
mU*mC*mU (SEQ ID NO: 2).
Any method herein described, wherein the
micrometastasis of the cancer is reduced by
at least 50% as compared to without adminis-
tering the pharmaceutical composition.
Any method herein described, wherein the
administering step is parenteral administra-
tion, including direct injection into a
tumor or its metastasis site by transcu-
taneous, intraarterial, intraductal,
intravenous, intradermal, intramuscular,
intraperitoneal, or subcutaneous
administration.
Any method herein described, wherein the
targeting sequence reduces the expression
of the TRAF3IP2 gene in the tumor by at
least 5-fold as compared to without the
targeting sequence for TRAF3IP2.
A method of treating a cancer having in-
crease in TRAF3IP2 expression comparing
to a normal cell, said method comprising
the steps of: a) administering a pharma-
ceutical composition into a subject be-
fore, during or in connection with at
least one of the following procedure:
surgery, diagnostic or therapeutic in-
tervention, chemotherapy, radiation
therapy, immunotherapy or targeted in-
tervention; wherein the pharmaceutical
composition comprises at least one
targeting sequence for TRAF3IP2 in a
pharmaceutically acceptable carrier in
an amount effective for the prevention
of micrometastasis.
Any method herein described, wherein
glycolytic activity of the cancer cells
is reduced by 50%.
Any method herein described, wherein
mTOR activation in the cancer cells is
inhibited
Any method herein described, wherein
NF-κB activity in the cancer cells is
inhibited.
Any method herein described, wherein
tumor growth is inhibited.
Any method herein described, wherein
the procedure in step (a) is chemo-
therapy, and wherein a chemotherapy
agent is used.
Any method herein described, wherein
the chemotherapy agent is selected
from: Actinomycin, All-trans
retinoic acid, Azacitidine,
Azathioprine, Bleomycin, Bortezomib,
Carboplatin, Capecitabine, Cisplatin,
Chlorambucil, Cyclophosphamide,
Cytarabine, Daunorubicin, Docetaxel,
Doxifluridine, Doxorubicin, Epirubicin,
Epothilone, Etoposide, Fluorouracil,
Gemcitabine, Hydroxyurea, Idarubicin,
Imatinib, Irinotecan, Mechlorethamine,
Mercaptopurine, Methotrexate,
Mitoxantrone, Oxaliplatin, Paclitaxel,
Pemetrexed, Teniposide, Tioguanine,
Topotecan, Valrubicin, Vemurafenib,
Vinblastine, Vincristine, and
Vindesine.
A method of sensitizing a tumor prior
to treating a patient with the tumor,
comprising the steps of: a) adminis-
tering a pharmaceutical composition
into a subject before at least one of
the following procedures for treating
the tumor: chemotherapy, radiation
therapy, immunotherapy or targeted
intervention, wherein the pharmaceu-
tical composition comprises at
least one silencing sequence for
TRAF3IP2 in a pharmaceutically
acceptable carrier in an amount
effective for sensitizing the tumor;
and b) performing the procedure for
treating the tumor.
Any method described herein, wherein
the procedure for treating the tumor
is chemotherapy.
Any method described herein, wherein
Paclitaxel, Temozolomide or Iguratimod
is used for the chemotherapy.
A method of determining malignancy of
a tumor in a patient, comprising the
steps of: obtaining a biological
sample from a patient; and detecting
presence of SEQ ID NO: 3-4 in the
biological sample; wherein the presence
of SEQ ID NO: 3-4 in the biological
sample indicates malignancy of the
tumor.
Any method described herein, wherein
the biological sample is a bodily fluid
sample.
Any method described herein, wherein
the tumor is breast cancer or
glioblastoma.
Any method described herein, wherein
the bodily fluid sample is blood sample.

As used herein, the term “expression vector” means a DNA or RNA into which a sequence of interest can be inserted that operably linked to a promoter such that the sequence will be transcribed or expressed from the promoter in the host cell/animal of interest. Thousands of such vectors are available. See e.g., Addgene.org which provides both a repository and a searchable database allowing vectors to be easily located and obtained from colleagues. See also Plasmid Information Database (PlasmID) and DNASU having over 191,000 plasmids. A collection of cloning vectors is also kept at the National Institute of Genetics as a resource for the biological research community. Furthermore, vectors (including particular ORFS therein) are usually available from colleagues.

As used herein, the term “targeting” refers to a nucleic acid sequence specifically complementary to and hybridize with at least a portion of a target gene.

As used herein, the term “increased TRAF3IP2 expression” refers to the level of TRAF3IP2 in a cancer cell being higher than a normal cell. In one embodiment, it refers to at least 20% increase of the level of TRAF3IP2. In another embodiment, it refers to at least 30%, 40%, 50%, 100%, 200% or 100% increase of the level of TRAF3IP2 comparing to a normal cell.

As used herein, the term “silencing” refers to the down-regulation of gene expression. At least 65%, 70%, 75%, 80% reduction should be achieved, but preferably, this term refers to the ability of a cell to prevent the expression of a certain gene. Gene silencing can occur during either transcription or translation and is often used in research and gene therapies.

As used herein, the term “suppressing” refers to down-regulating gene expression by at least 30%, and preferably at least 40%, 45%, 50%, 55% or 60% down-regulation should be achieve.

By “preventing” gene expression, we mean no detectable intact gene expression is detected when assayed by Northern blot using a radioactively end-labeled oligomer that is complementary to the gene being silenced. Nonetheless, there may be minute amounts of expression that could be detected by extremely sensitive methods.

The term “silencer” as used herein refers to an exogenous sequence that can be introduced into cells and used to silence gene expression in that cell. There are several different types of silencers, including at least antisense oligonucleotides, ribozymes, RNA interference, and the like. Genes can be silenced by e.g., dsRNA that decomposes mRNA, siRNA molecules that cause the endonucleatic cleavage of the target mRNA molecules or by miRNA molecules that suppress translation of the mRNA molecule or by shRNA, as well as by endoribonuclease-prepared siRNAs (esiRNAs), which are a mixture of siRNA oligos resulting from cleavage of long double-stranded RNA (dsRNA) with an endoribonuclease such as Escherichia coli RNase III or dicer. The term “silencer” is not limited to any one particular methodology, unless so specified.

The term “inhibit” or inhibition refers to the down-regulation of the expression of a target gene or the reduction of a phenotype, by at least 10%, or at least 33%, or at least 50%, or at least 80% of its gene expression relative the control.

By “exosomes” what is meant herein are cell-derived vesicles that are present in many and perhaps all biological fluids, including blood, urine, and cultured medium of cell cultures.

As used herein, the expressions “cell”, “cell line” and “cell culture” are used interchangeably and all such designations include progeny. Thus, the words “cells” and similar designations include the primary subject cell and cultures derived therefrom without regard for the number of transfers. It is also understood that all progeny may not be precisely identical in DNA content, due to deliberate or inadvertent mutations that arise after genetic engineering is concluded. Mutant progeny that have the same function or biological activity as screened for in the originally transformed cell are included. Where distinct designations are intended, it will be clear from the context.

The term “sensitizing” refers to the synergistic induction of apoptosis in tumor cells by combining different therapies. For example, by modulating the DNA repair pathways inside the tumor cells, it is possible to sensitize cancer cells.

The terms “operably associated” or “operably linked,” as used herein, refer to functionally coupled nucleic acid sequences.

As used herein “recombinant” is relating to, derived from, or containing genetically “engineered” material. In other words, the genome was intentionally manipulated by the hand of man in some way.

“Reduced activity” or “inactivation” is defined herein to be at least a 75% reduction in protein/gene activity, as compared with an appropriate control species. Preferably, at least 80, 85, 90, 95% reduction in activity is attained, and in the most preferred embodiment, the activity is eliminated (100%). Proteins can be inactivated with inhibitors, by mutation, or by suppression of expression or translation, and the like. A negative superscript, as in ACT1⁻, indicates reduced activity.

As used herein, “pharmaceutically acceptable carrier” refers to any carrier that is capable of delivering oligonucleotide to target cells. Examples of the pharmaceutically acceptable carrier include, but not limited to, nucleic acid carrier, cationic lipids, peptide-mediated carrier such as cell-penetrating peptides, nanogel carrier, liposomes, small molecule tags (including cholesterol-modification, membrane-permeant peptides, folate, antibiotics, VITE, and VITA), and cationic polymers.

As used herein, “biological sample” refers to samples taken from a subject, including but not limited to bodily fluids, tissue, bone marrow, cell lines, exhaled air, feces, hair, nail, etc. The term “bodily fluids” refers to blood, urine, saliva, sweat, semen, mucus, vaginal fluids, fluids from cytology (ascites, pleural fluid, synovial fluid, etc.).

As used herein the specification, “a” or “an” may mean one or more. As used herein in the claim(s), when used in conjunction with the word “comprising”, the words “a” or “an” may mean one or more than one. As used herein “another” may mean at least a second or more.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

Wherever any of the phrases “for example,” “such as,” “including” and the like are used herein, the phrase “and without limitation” is understood to follow unless explicitly stated otherwise. Similarly, “an example,” “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The disclosure may use one or more of the following abbreviations:

| Abbreviation | Meaning |
|---|---|
| ASO | Anti-sense Oligonucleotides |
| BC | Breast cancer |
| bi-shRNA | bifunctional shRNA |
| esiRNA | Endoribonuclease-prepared siRNAs |
| GBM | Glioblastoma |
| GFP | Green fluorescent protein |
| KD | Knock down (refers to silencers herein) |
| miRNA | microRNA |
| MSC | Mesenchymal stem cells |
| NK | Natural killer |
| RFP | Red fluorescent protein |
| RNAi | RNA interference |
| shRNA | Small hairpin RNA |
| siRNA | Small interfering RNAs |
| TAM | Tumor associated macrophages |
| TILs | Tumor infiltrating lymphocytes |
| TNBC | Triple negative breast cancer |
| TRAF3IP2 | TRAF3-INTERACTING PROTEIN 2 aka NUCLEAR FACTOR |
| | KAPPA-B ACTIVATOR 1 or ACT1 (UniProt O43734) |
| TME | Tumor microenvironment |

### BRIEF DESCRIPTION OF THE DRAWINGS

FIG. 1. Targeting TRAF3IP2 reduces cell proliferation, angiogenesis and metastasis.

FIG. 2A-B. TRAF3IP2 expression in breast cancer cells and tissues.

FIG. 3A. Subcutaneous treatment of tumor in PDX model between TRAF3IP2 shRNA-LV and SCR shRNA-LV.

FIG. 3B. Intraperitoneal treatment of tumor in PDX model between TRAF3IP2 shRNA-LV and SCR shRNA-LV.

FIG. 4A. Detection of micrometastasis based on human-specific alpha-satellite region on chromosome 17.

FIG. 4B. Detection of Micrometastasis based on estimated number of human cells detected in a total of 4×10⁴ cells.

FIG. 4C-D. Exosome characterization between subcutaneous treatment and intraperitoneal treatment of TRAF3IP2 shRNA-LV/SCR shRNA-LV/no treatment.

FIG. 4E. Detection of micrometastasis using PCR. Key TNBC metastatic niches were analyzed for micrometastasis using primers directed towards a human-specific alpha-satellite DNA sequence of the centromere region of human chromosome 17. No human specific DNA sequence was detected in the most common metastatic sites in TRAF3IP2-AO treated animals.

FIG. 5A-B. Silencing TRAF3IP2 in tumors reduces tumor size.

FIG. 6. Silencing TRAF3IP2 inhibits malignant metabolism in TNBC cells.

FIG. 7. A) RT-qPCR revealed the expression of genes related to inflammation, angiogenesis, cell cycle and apoptosis, and exosome release in tumors of TRAF3IP2-AO treated animals (vs. SCR-AO). B) Western Blotting Analysis of tumor lysates demonstrates significantly reduced levels of pro-tumorigenic proteins in animals treated with TRAF3IP2-AO. C, D) Immunohistochemical analysis of tumor tissue revealed reduced expression of pro-tumorigenic proteins (C) and human-specific mitochondrial markers in TRAF3IP2-AO treated animals (vs. SCR-AO; scale bar 100 μm). GAPDH: 36 kDa; TRAF3IP2: 65 kDa; IL8: 11 kDa; NFkB: 60 kDa; VEGF: 25 kDa.

FIG. 8. Effect of targeting TRAF3IP2 on chemoresistance.

FIG. 9. Treatment of late stage metastatic TNBC.

FIG. 10. Effect of TRAF3IP2 silencing on angiogenesis.

FIG. 11. Effect of targeting TRAF3IP2 using antisense oligonucleotide on angiogenesis in vivo.

FIG. 12. TRAF3IP2 expression in glioblastoma cells and tissues.

FIG. 13. Silencing TRAF3IP2 suppresses glioblastoma sumorigenesis.

FIG. 14. Targeting TRA3IP2 by Antisense Oligonucleotide suppresses glioblastoma growth.

FIG. 15. Detection of Intracranial Dissemination.

FIG. 16. Silencing of TRAF3IP2 enhances the effect of igurotimod on glioblastoma and on triple negative cancer cell growth.

FIG. 17. Targeting TRAF3IP2 causes change in anti-tumor immune response.

### DETAILED DESCRIPTION OF THE DISCLOSURE

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

Furthermore, while the invention is exemplified in breast cancer cell lines injected into mice with particular vectors and silencers, this is for proof of concept only, and the methods are expected to work in many different tumors with a variety of silencer delivery methods and with a variety of silencer sequences.

TRAF3IP2 mediates IL-17-dependent NF-κB activation and inflammatory signaling, suggesting its potential in BC development and progression. TRAF3IP2 is also an upstream regulator of JNK, a stress-activated protein kinase involved in AP-1 activation, suggesting that increased TRAF3IP2 expression enhances inflammation via NF-κB- and AP-1-dependent mechanisms. By regulating inflammatory signaling, TRAF3IP2 plays a crucial role in TME formation. The TME, a highly dynamic space evolved in relation to tumor over time, regulates bidirectional communication between malignant and adjacent non-malignant cells, including neoangiogenesis and malignant metabolism. High energy demand of BC cells enhances glycolytic flux particularly in a harsh hypoxic environment. BC cells exploit oxidative phosphorylation pathways to dynamically adapt its metabolic processes to TME. Rapid tumor growth creates a hypoxic TME, activates HIF1-α expression, increases VEGF levels, and promotes neoangiogenesis, thus facilitating further tumor growth and metastasis. Activation of HIF-1α also drives the metabolic transition to aerobic glycolysis. Therefore, metabolic alterations in cancer make significant contributions to the metastatic phenotype and neoangiogenesis.

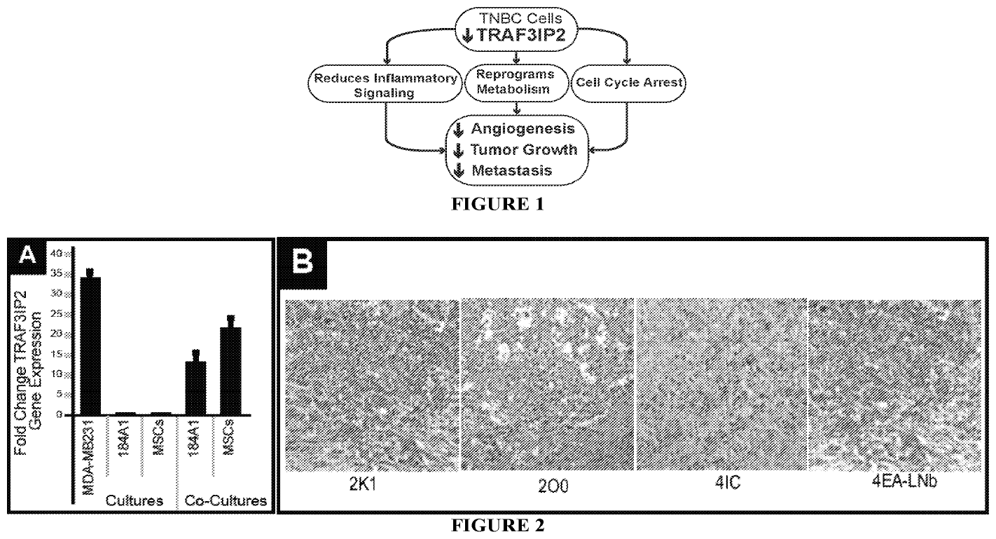

The data shows that targeting and silencing TRAF3IP2 decreases aerobic glycolysis, leading to reduced neoangiogenesis and metastasis, thus strongly linking increased TRAF3IP2 expression to metabolic alterations and neoangiogenesis, tumor growth and metastasis. See FIG. 1. The preliminary data also indicate that silencing TRAF3IP2 promotes BC cell senescence. Senescence induces an inflammatory response and inflammatory cell infiltration. Canonical senescence is precipitated by nuclear damage and results in a senescence-associated secretory phenotype (SASP), and is associated with the secretion of proteases, cytokines, growth factors, and pro-angiogenic mediators.

### TRAF3IP2 Causes Tumorigenesis in Breast Cancer In Vitro and In Vivo

Analyzing the TRAF3IP2 expression level in different cells shows significantly increased level of TRAF3IP2 in primary human breast cancer tissue, see FIG. 2(A). FIG. 2(B) shows representative sections from four independent “Triple Negative Breast Cancer” (TNBC) tissues, wherein increased TRAF3IP2 expression (brown) is stained by IHC, counterstained with hematoxylin (blue).

Female 4-6 weeks old NSG mice were injected intra-mammary with MDA_{TRAF3IP2 KD} Or 4IC_{TRAF3IP2 KD} cells (1×106 cells in PBS and Martigel) and compared to control group (MDA_{Control shRNA} or 4IC_{Control shRNA})(n=10/group). Due to large tumor growth in animals injected with MDA_{Control shRNA} or 4IC_{Control shRNA} cells, they were euthanized 10 and 4 weeks post-injection, respectively (AI & BI). Significant gross metastasis was found in various tissues. The extent of metastasis in abdomen, liver, kidneys and lungs (arrows) of MDA_{Control shRNA} or 4IC_{ControlshRNA} injected animals are shown (AI &BI).

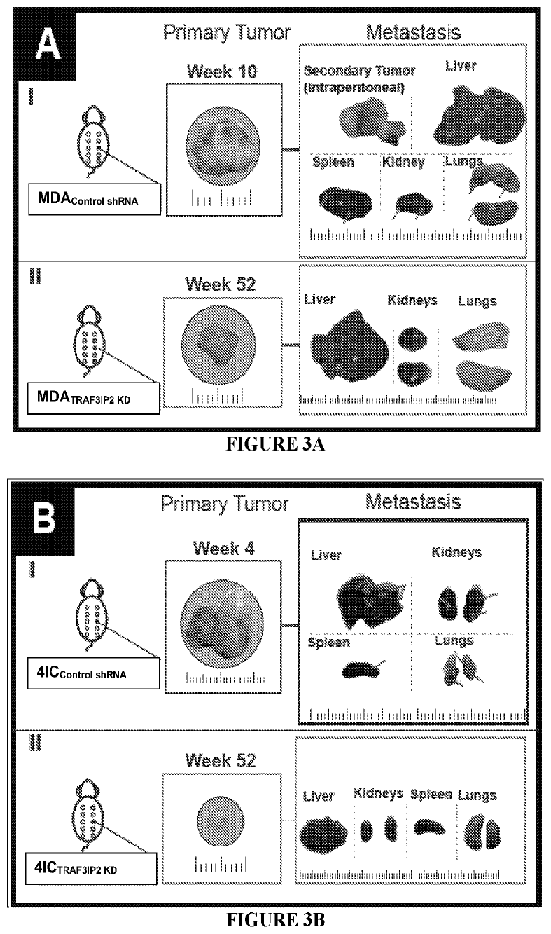

Both MDA_{TRAF3IP2 KD} or 4IC_{TRAF3IP2 KD} injected animals showed minimal tumor growth upon euthanasia after week 52 (AII and BII). Further, no metastasis was detected in liver, kidneys and lungs in both groups. In other words, administration of TRAF3IP2-silenced TNBC cell line (MDA_{TRAF3IP2KD}) and TNBC primary cells (4IC_{TRAF3IP2KD}) into a mouse mammary fat pad prevents tumor growth, inhibits metastasis, and prolongs survival (FIG. 3A-II and 3B-II).

The following materials were used herein:

| | |
|---|---|
| MDA-MB231 cells | A human breast cancer cell line, available from Sigm-Aldrich ® |
| MDA_{KDTRAF3IP2} cells | MDA-MB231 cells transformed with a lentiviral vector encoding a |
| | TRAF3IP2 silencer. |
| MDA_{KDRab27} cells | MDA-MB231 cells transformed with a lentiviral vector encoding RAB27A |
| | silencer |
| 184A1 cells | A human mammary gland cell lines, established by chemical |
| | transformation (ATCC ® CRL-8798) |
| Lentiviral vector | A lentiviral-based vector (e.g. pLKO.1-puro or pLKO.1-puro-CMV- |
| | TurboGFP ™), preferably having a transactivator inducible promoter, such |
| | as IL1B promoter which will be activated in presence of excessive |
| | amounts of IL1B within tumor microenvironment. |
| 4IC | Primary TNBC cell line |

### Micrometastasis Detection

Genomic DNA from samples was extracted using the QIAmp DNA Mini Kit (Qiagen). Presence of human DNA was demonstrated using Quantitative Real-Time PCR, detecting a human-specific α-satelite region on chromosome 17. The RT-PCR reaction mixture contained 250 ng genomic DNA template, 250 nM of each primer and iTaq™ Universal SYBR® Green Supermix (BioRad) following the manufacturer's protocol. An initial DNA denaturation and Taq activation at 95ºC for 5 min was followed by 35 one-minute cycles of denaturation at 95° C., annealing at 57° C. and extension at 72° C. Each sample was tested in triplicate.

Amplified DNA fragments were analyzed by electrophoresis through 1% agarose gel, followed by ethidium bromide staining and visualization through ultraviolet light (BioRad ChemiDoc Imaging System) to assure the correct band size. Genomic DNA samples from human 293T cells served as the positive control, NSG mouse liver tissue as the negative control. Several dilutions of human in mouse DNA created a calibration curve.

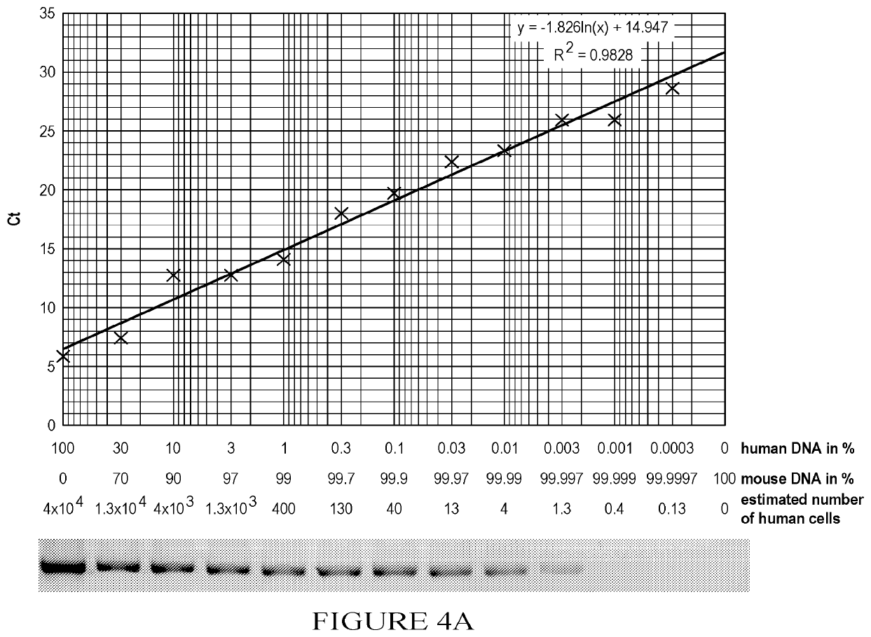

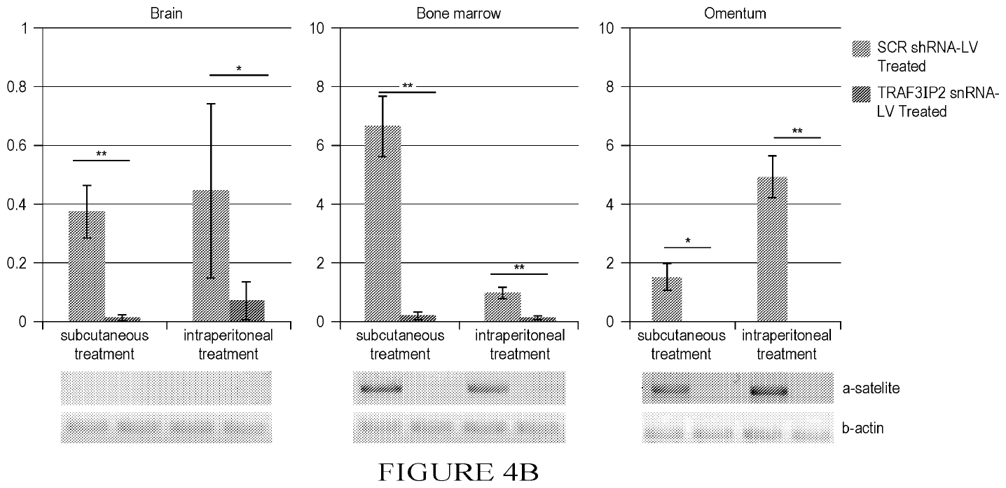

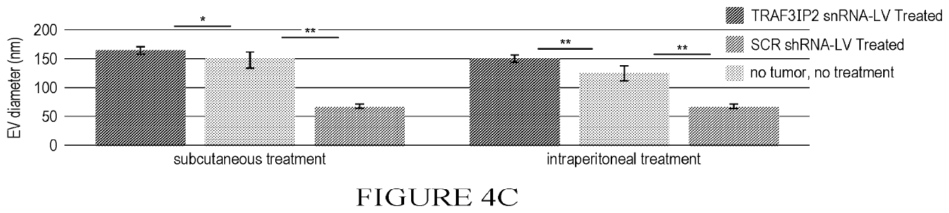

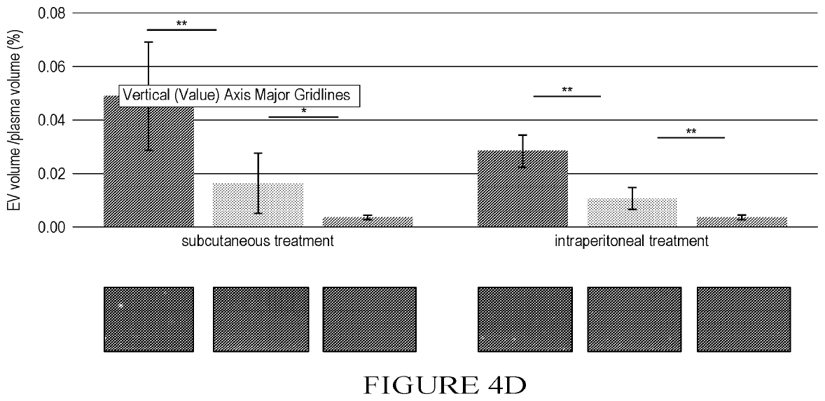

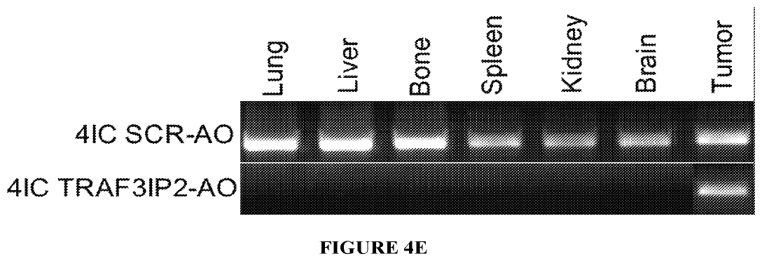

Human-specific α-satellite region on chromosome 17 is used to distinguish human DNA from mouse DNA. As shown in FIG. 4A, genomic DNA samples from human 293T cells served as the positive control, NSG mouse liver tissue as the negative control. Several dilutions of human in mouse DNA created a calibration curve.

With an assumption of 6 pg DNA per cell, roughly 40000 cells were analyzed for human origin in the 250 ng DNA template. The dilution of 0.002% human DNA is therefore equivalent to one cell human cell in the template, represented by a Ct value of 26.3. Following that division, Ct values under 26.3 demonstrate definite human cells and can be interpreted as micrometastasis in the respective analyzed tissue.

2/3 mice were analyzed. Mice euthanized on day 29 post tumor induction (all animals of control group, 3/6 animals of treatment group) Limited amount of animals available due to FFPE fixation of organs from 3/6 mice. FIG. 4B shows the estimated number of human cells detected in a total of 4×10⁴ cells. As can be seen, TRAF3IP2 shRNA-LV treated mice show significantly lower human DNA in brain, bone marrow or omentum.

Additionally, exosome characterization was used to determine metastatic abilities of the tumors in different treatment groups. Exosomes are the main insoluble components of the tumor microenvironment. Exosomes are small membranous extracellular vesicles (40-140 nm in diameter) that are released in extracellular space. In addition to production by tumor cells, exosome-like vesicles are produced by various non-malignant cell types. Structurally, these vesicles consist of a lipid bi-layer membrane similar to the cellular membrane, proteins, including host specific proteins, mRNA, microRNA (miRNA) and transcription factors.

Exosomes can affect various cell types by transferring their content to various cells. The growing interest in the characterization of exosome-like vesicles in cancer research arises from their potential role in carrying a large array of oncogenic elements released by malignant cells, such as oncogenic proteins and miRNAs. Such oncogenic proteins and miRNAs can traverse the tumor microenvironment and can be taken up by recipient non-malignant cells; this can result in the transfer of oncogenic activity.

Nanoparticle tracking analysis-based exosome characterization utilizes the properties of both light scattering using a light microscope (composition), and Brownian motion of individual vesicle is tracked by software (size) in order to obtain the particle size distribution of samples in liquid suspension. Total concentration is also calculated.

Analysis of EV extracted from plasma in different groups showed that EV of the TRAF3IP2 shRNA-LV treated groups were significantly larger than those of the control group, measured by mode diameter and volume of particles. EV of all PDX groups, no matter what treatment received, were significantly larger than EV of untreated NSG control mice without any induced tumor (FIG. 4C).

The concentration of particles per mL was significantly lower in SCR shRNA-LV treated mice, compared to both the TRAF3IP2 shRNA-LV treated animals (sc. injections p=0.0166; ip. injections p=0.0002) and the untreated control group without tumor (sc. injections p=0.0024; ip. injections p<0.0001).

Combining data on EV size and concentration results in reporting the fraction of EV in total plasma volume. In subcutaneous TRAF3IP2 shRNA-LV treated mice EV constitute 0.048% of plasma volume, whereas in SCR shRNA-LV treated animals the volume of EV determines only 0.016% (p=0.0097). See FIG. 4D. Groups of intraperitoneal injections show a similar trend: EV of TRAF3IP2 shRNA-LV treated mice define 0.028% of plasma volume, whereas the number drops to 0.010% (p=0.0003) in the SCR shRNA-LV treated animals. The lowest percentage (0.003%) is observed in untreated NSG mice without tumor and is considered as the baseline of EV release.

Results therefore indicate an increased level of apoptotic cells in TRAF3IP2 shRNA-LV treated animals, represented by larger plasma EV.

Moreover, PCR using primers that specifically detect human-specific a-satellite DNA sequence of the centromere region of human chromosome 17 indicated no micrometastasis in the treated group. In contrast, SCR-AO treated mice showed metastasis in lung, liver, bone, kidney and spleen.

### Silencing TRAF3IP2 in Triple-Negative Breast Cancer PDX Model

The effects of targeting and silencing TRAF3IP2 on tumor growth and metastasis of patient-derived TNBC tissues (TNBC PDX cells (TU-BcX-49S, TU-BcX-4QAN, TU-BcX-56S) in PDX models was evaluated. The results of this study determines whether silencing TRAF3IP2 will suppress tumor growth, inhibit metastasis, and extend survival of PDXs.

24 animals developed tumor, and were divided in 4 treatment groups. Animals of the control group needed to be sacrificed on Day 29, due to extremely large tumors that impaired movement. Survival Curve was created as Kaplan Meyer based on the Date of Death of the animals. Animals were euthanized when condition and/or tumor size made it necessary. Censored animals also included: TRAF3IP2 KD animals sacrificed at same day as Control for Pathology study.

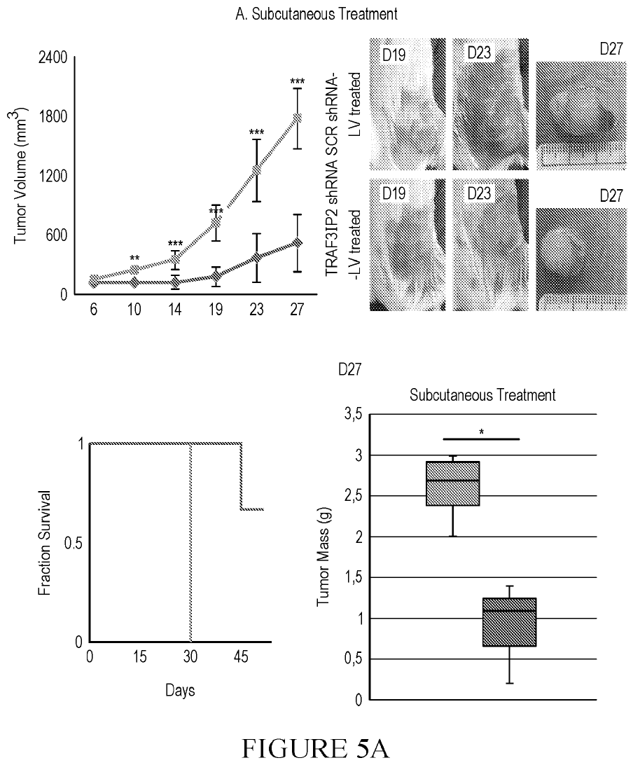

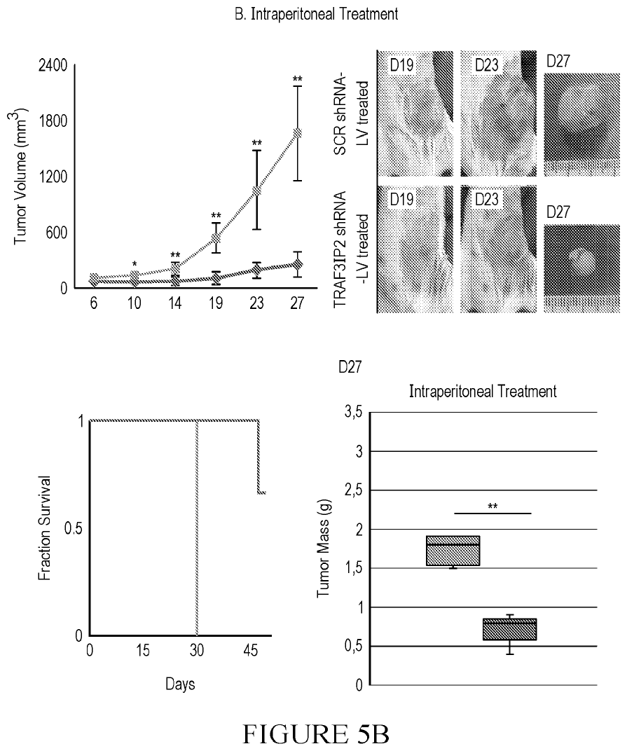

FIGS. 5A-B display the fraction survival over time for the Subcutaneos and Intraperitoneal Treatment. Tumor mass Day 29 post surgery was measured (measurements at the same time for all treatment groups).

The results were compared based on the method of delivery between Subcutaneous (SC) and Intraperitoneal treatment (IP). Average tumor reductions for SC treatment is 65.3%, whereas average tumore reduction for IP Treatment: is 60%

For max tumor reduction, SC Treatment achieved a 92.3% reduction, whereas IP Treatment was 77.1%. Therefore, it is clearly shown that in PDX model, targeting and silencing TRAF3IP2, regardless of method of delivery, successfully reduces tumor size.

### Effects of Silencing TRAF3IP2 on Malignant Metabolism in Breast Cancer

In vitro and in vivo assays are designed for generation of total ROS and superoxide in BCTRAF3IP2 KD cells, to assess the levels of NADPH oxidase 2 (Nox2), a potent producer of superoxide. To confirm the role of superoxide in TRAF3IP2-silencing mediated mitochondrial damage, the cells were treated with a free radical scavenger (i.e. glutathione) and investigate whether (a) mitochondrial damage can be prevented and (b) mitochondrial function can be restored.

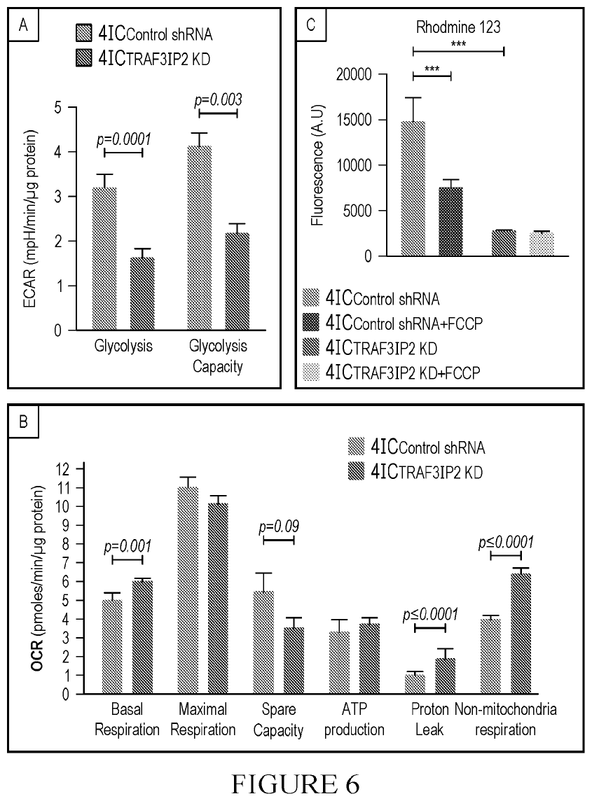

FIG. 6(A) shows Glycolysis analysis in 4IC_{TRAF3IP2 KD} VS. 4IC_{control shRNA} cells. As seen in FIG. 6(A), silencing TRAF3IP2 inhibits glycolytic activity by 50%. FIG. 6(B) shows the results of Seahorse assay to assess the effects of TRAF3IP2 silencing on mitochondrial function. Results indicate significant changes in TNBC metabolism following TRAF3IP2 silencing. FIG. 6(C) shows the results of membrane potential assay indicates that silencing TRAF3IP2 decreases membrane potential, implying mitochondrial damage. Notably, the addition of FCCP, a potent uncoupling agent, does not decrease mitochondrial membrane potential, further suggesting that TRAF3IP2 knockdown is highly effective in reducing membrane potential and thus inducing mitochondrial dysfunction.

### Effects of Silencing TRAF3IP2 on Inflammatory Signaling in Breast Cancer

As shown above, targeting and silencing TRAF3IP2 significantly reduces NF-κB activation, inhibits expression of inflammatory cytokines (such as IL-6 and IL-8), and reduces mTOR activation. The PI3K/AKT/mTOR pathway has been shown to: (1) phosphorylate and degrade IκB (inhibitor of KB), thus activating NF-κB and inflammation and (2) activate HIF-1α and subsequently results in VEGF-driven angiogenesis. It is therefore proposed that targeting TRAF3IP2 in TNBC samples would coordinately suppresses inflammation, downstream pro-tumorigenic metabolism, angiogenesis, and tumor growth in the PDX model. The inflammatory signaling pathway is studied in BC_{TRAF3IP2 KD} VS. BC_{control shRNA}.

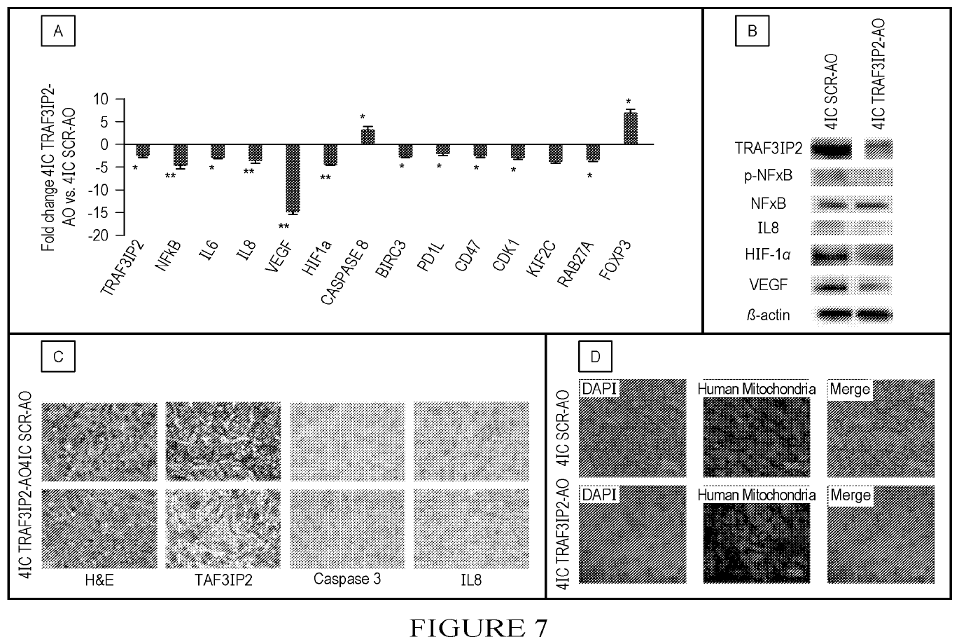

In FIG. 7(A) RT-qPCR revealed the expression of genes related to inflammation, angiogenesis, cell cycle and apoptosis, and exosome release in tumors of TRAF3IP2-AO treated animals (vs. SCR-AO). In FIG. 7(B), Western Blotting Analysis of tumor lysates demonstrates significantly reduced levels of pro-tumorigenic proteins in animals treated with TRAF3IP2-AO. In FIG. 7(C, D), Immunohistochemical analysis of tumor tissue revealed reduced expression of pro-tumorigenic proteins. FIG. 7(C) shows human-specific mitochondrial markers in TRAF3IP2-AO treated animals (vs. SCR-AO; scale bar 100 μm).

Post-mortem analysis of residual tumor confirmed that targeting TRAF3IP2 suppresses inflammation, angiogenesis, cell cycle and apoptosis, and genes involved in exosome release (FIG. 7(A)). Protein expression analysis showed that tumors treated with TRAF3IP2-AO displayed significantly lower levels of TRAF3IP2, IL8, IL1b, and VEGF. Together, these molecular data confirm the crucial role of TRAF3IP2 in malignant inflammation and angiogenesis, and strongly suggest the mechanistic involvement of TRAF3IP2 in regulating multiple protumorigenic pathways (FIG. 7(B)). In addition, immunohistochemical analysis showed decreased levels of TRAF3IP2 and IL8 and increased Caspase 3, which is associated with tumor regression in the treated group (FIG. 7(C)). Analysis of residual mass indicated that cells from the host had infiltrated the tumor site forming the greatest majority of the cells (FIG. 7(D)), whereas human breast cancer cells were sparse (negative for human-specific mitochondrial marker; FIG. 7(D)).

### Silencing TRAF3IP2 and Chemoresistance

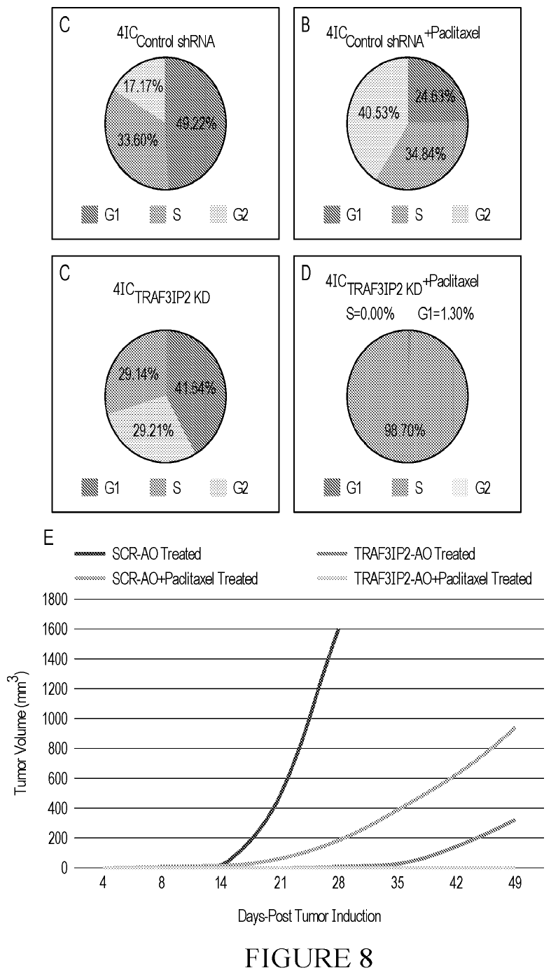

Whether silencing TRAF3IP2 would successfully reduce tumor growth, combining TRAF3IP2 silencer with chemotherapy agents may have improved or synergistic effect on tumor treatment. One chemotherapy agent being tested is Paclitaxel. Preliminary results show that targeting TRAF3IP2 enhances the efficacy of Paclitaxel in TNBC cells, resulting in maximally suppressed tumor growth. In that experiment, 4IC were isolated from a TNBC patient that showed limited response to chemotherapy, silenced for TRAF3IP2 (4ICTRAF3IP2 KD; cells transduced with scrambled shRNA served as a control, 4ICControl shRNA), treated with Paclitaxel and analyzed for cell cycle profile in vitro. Paclitaxel has been previously shown to arrest cell cycle progression at the G2/M phase, resulting in apoptosis. Our data show that Paclitaxel treatment alone increased the proportion of cells in G2 by 23%, as compared to a 12% increase from TRAF3IP2 knockdown alone. Importantly, targeting TRAF3IP2 reduced the proportion of cells in S phase by 3%. Notably, combining paclitaxel with TRAF3IP2 knockdown completely prevented entry into S phase, and arrested cells in the G2/M checkpoint (FIG. 8A-D).

We next examined whether the combination therapy (Paclitaxil+TRAF3IP2 knockdown) is equally effective in vivo. At first, tumors were induced using wild type 4IC cells using a similar set up described in FIG. 5. After tumor induction, each group was treated with one of the following: SCR-AO (4IC SCR-AO Treated), SCR-AO and Paclitaxel (SCR-AO+Paclitaxel Treated), TRAF3IP2-AO Treated, or TRAF3IP2-AO and Paclitaxel (TRAF3IP2-AO+Paclitaxel Treated). Animals in the SCR-AO group had to be euthanized by day 28 due to increased tumor burden. Paclitaxel reduced tumor volume by 42% and targeting TRAF3IP2 reduced tumor size by 78%. Their combination totally eliminated the tumor (FIG. 8E). These clinically relevant in vitro and in vivo data indicate that combining paclitaxel with TRAF3IP2 knockdown is superior and will overcome the challenges associated with cancer cell responses to standard chemotherapies. In other words, the COMBINATION OF

Sensitization of Tumor Cells with TRAF3IP2 Silencing

The targeting and silencing of TRAF3IP2 not only prevents the growth of a tumor and prevents metastasis and micromestastasis, but it also sensitizes tumors in a synergistic way to the treatment with other cytostatic, cytotoxic, oncolytic or immunmodulatory drugs or radiation treatment. An example of these drugs or treatments are for example Paclitaxel, Temozolomide, Iguratimod plus additional or without additional radiation treatment.

Indeed as shown above, the combination of TRAF3IP2 silencing and administration of Paclitaxel can completely eliminate the tumor as shown in FIG. 8E. It is therefore possible to silence TRAF3IP2 in the tumors first, followed by other cytostatic, cytotoxic, oncolytic, immunomodulatory agents or radiation treatments.

Additionally, Iguratimod (N-[7-(methanesulfonamido)-4-oxo-6-phenoxychromen-3-yl]formamide, IGU), a small drug, can be used as a potent anti-inflammatory molecule. IGU has various mechanisms of actions including disrupting IL-17R pathway, and effectively impairs the interaction between TRAF5 and TRAF3IP2. It also has well established capacities of minimizing TRAF3IP2-mediated NF-κB activation. We therefore hypothesized that IGU could be utilized as a TRAF3IP2-suppressing agent that would reduce malignant tumor growth and metastasis in TNBC and GBM cells.

In vitro investigations were carried out using transduced TNBC cell line (MDAMB-231) and patient derived TNBC cells (4IC), pediatric GBM cell line (KNS), and adult GBM cell line (U87) by targeting and suppressing/silencing TRAF3IP2 expression using shRNA Lentiviral vector (TRAF3IP2KD) versus shRNA control (SCR). Using MTT assay, we treated malignant cells with low and high concentrations of IGU to test cell proliferation, viability, and apoptosis compared to vehicle (DMSO) treated cells. It is shown in FIG. 16 that the silencing of TRAF3IP2 enhances the effect of IGU on adult glioblastoma cells (U87), pediatric glioblastoma cells (KNS) and on triple negative cancer cell growth (data not shown).

Particularly regarding U87, at 24 hours post treatment, the two concentration groups in U87 and U87_{TRAF3IP2KD} show similar cell viability. At 48 hours, in C2 (224.823 mM IGU) the control group has about 50% viability, whereas U87_{TRAF3IP2KD} has about 40% viability. Similarly at 72 hours, in C2 the control group has about 40% cell viability, whereas in U87TRAF3IP2KD has about 20% cell viability.

Regarding KNS, at 24 hours post treatment, in C2 the KNS control has about 65% cell viability, whereas in KNS_{TRAF3IP2KD} it shows about 55% cell viability. At 48 hours post treatment, in C1 the KNS control has about 55% cell viability, whereas in KNS_{TRAF3IP2KD} it shows about 45% cell viability. At 72 hours post treatment, in C2 the KNS control shows about 40% cell viability, whereas in it shows about 25% cell viability.

Taken together, it is shown that targeting and suppressing/silencing TRAF3IP2 expression indeed enhances IGU's effect on glioblastoma and triple negative breast cancer cells.

### Treatment of Late Stage Triple-Negative Breast Cancer

To see if targeting and silencing TRAF3IP2 eliminates metastatic tumors in advanced metastatic TNBC, PDX model were used. 4IC tumors were induced intramammary into mice using 4IC cells [carrying a luciferase-expressing gene for ‘bioluminescence imaging’. Treatment was initiated in late stage BC indicated by advanced metastasis detectable by luciferase imaging (day 42). Animals were treated with TRAF3IP2-AO (100 μg in 50 μl) administered interperitoneally every 48 hours. Control group received SCR-AO in the same fashion (Data not shown). Representative images showing the extend of metastasis after treatment 1(T1), T5, T10, and T15. The control animals were treated with SCR-AO only on T1 and were euthanized due to the increasing tumor growth.

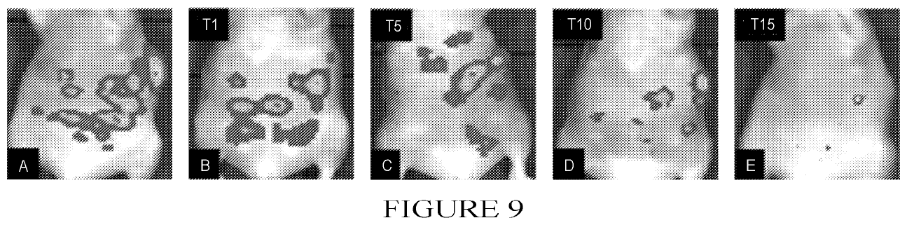

The results indicate a marked reduction of luciferase signal, reaching undetectable levels after 15 treatments (FIG. 9). This is evidence that solid tumors may be treated or eliminated by targeting and silencing TRAF3IP2.

### Effects of Silencing TRAF3IP2 on Angiogenesis in Breast Cancer

Increased angiogenesis contributes to reduced patient survival. NF-κB is involved in angiogenesis in breast cancer. Blockade of NF-κB signaling significantly inhibits TNBC growth and angiogenesis through reduction of VEGF and IL-8. However, sole targeting of NF-κB in a clinical setting yielded disappointing results. We reported that targeting and silencing TRAF3IP2 expression decreases VEGF expression, and inhibits 1) angiogenesis in vitro and 2) neoangiogenesis in vivo. In vivo data indicate that treating tumors have reduced mTOR-driven HIF-la activation and downstream VEGF driven neovascularization. Therefore, we hypothesize that TRAF3IP2 is a key coordinator of neoangiogenesis in BC.

Therefore, the effects of targeting and silencing TRAF3IP2 on angiogenesis in BC PDX models is studied and compared with tumors induced by BCTRAF3IP2 KD and BC_{control shRNA} cells for each primary BC specimen. Effect of TRAF3IP2 silencing on angiogenesis: HBEC cells were incubated with conditioned media from 4IC_{KDTRAF3IP2} and 4IC_{controlshRNA} cultures. The level of tube formation is shown in HBEC cells incubated with 4IC_{controlshRNA} conditioned media (MDAControl shRNACM) (A) and conditioned media from MDA_{TRAF3IP2KD} cultures (MDA_{TRAF3IP2KD}CM). There is significantly fewer tube formation in HBEC cells incubated with 4IC_{TRAF3IP2KD}CM (B). The angiogenesis is restored when recombinant VEGF was added to the 4IC_{TRAF3IP2KD} conditioned media (C and D). The graph shows the total tube formation, total branching points, loops, and total net in HBEC incubated with each 4IC_{KDTRAF3IP2}CM and 4IC_{controlshRNA}CM, and addition of VEGF to each conditions (E).

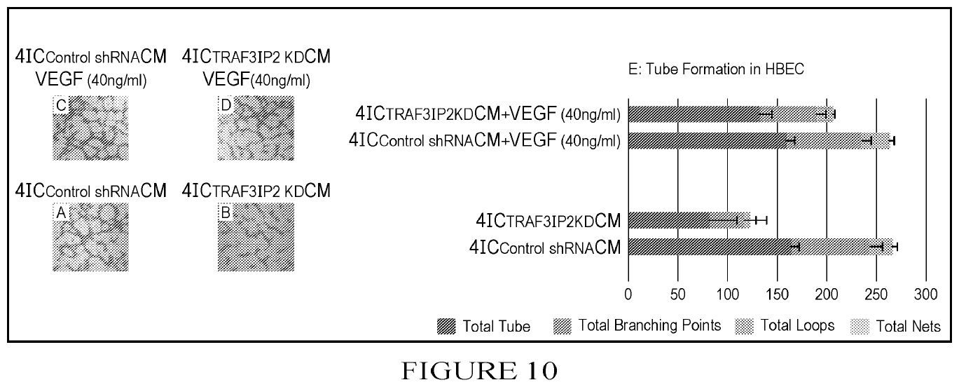

As shown in FIG. 10, primary human breast epithelial cells (HBEC) display increased angiogenesis when treated with conditioned media from 4ICcontrol shRNA cultures. However, the HBEC angiogenesis is significantly reduced when incubated with conditioned media from 4IC_{TRAF3IP2KD} (4IC_{TRAF3IP2KDCM}). In addition, angiogenesis is restored when recombinant VEGF is added to HBEC cells incubated with 4IC_{KDTRAF3IP2CM}.

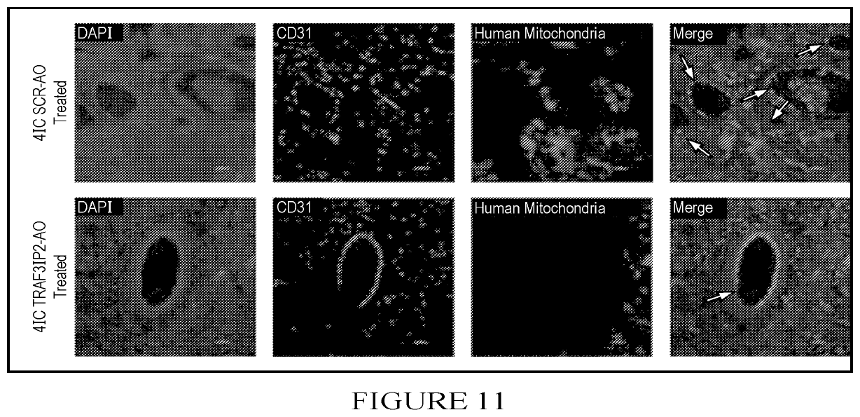

Further, in vivo data indicate that treating TNBC with TRAF3IP2-AO results in reduced tumor growth. Immunohistochemical analysis of the vessel structure within the TRAF3IP2-AO treated tumors revealed lower blood vessel density/angiogenesis in TRAF3IP2-AO treated tumors (Arrows in FIG. 11), indicated by reduced CD31 staining (an epithelial marker) compared to SCR-AO treated tumors. In addition, staining for human-specific mitochondria showed significantly less number in TNBC cells (human cells) in residual TRAF3IP2-AO treated tumor tissue. This demonstrates a critical role for TRAF3IP2 in neoangiogenesis. Also, it is notable that TRAF3IP2 induces the expression and release of pro-angiogenic mediators into the TME, activating endothelial cells to promote neo-vascularization. TRAF3IP2 silencing.

### TRAF3IP2 Expression in Glioblastoma

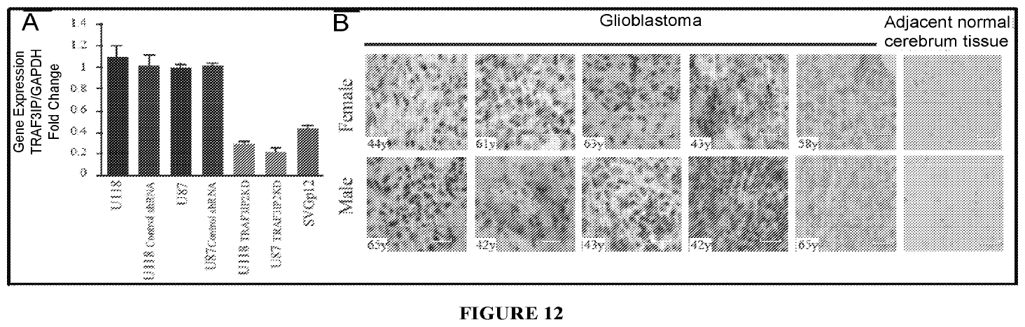

Expression of TRAF3IP2 in U87, U118 and SVGp12 was analyzed and shown in FIG. 12. The expression of TRA3IP2 is significantly higher compared to SVGp12, a non-malignant glial cell line. TRAF3IP2 mRNA expression in TRAF3IP2 knockdown in U87 and U118 cells was compared to the control cells (U118control shRNA, U87, U87control shRNA, U118TRAF3IP2 KD, U87TRAF3IP2 KD) and SVG p12 cells by RT-qPCR. Results were normalized to values obtained in U87 and U118 cells respectively (n=9/cell type; P<0.05). B) Representative sections from glioblastoma tissues from ten patients show increased TRAF3IP2 expression (brown) by IHC, counterstained with hematoxylin (blue). Scale bar, 100 μm.

### Silencing TRAF3IP2 Suppresses Glioblastoma Tumorigenesis

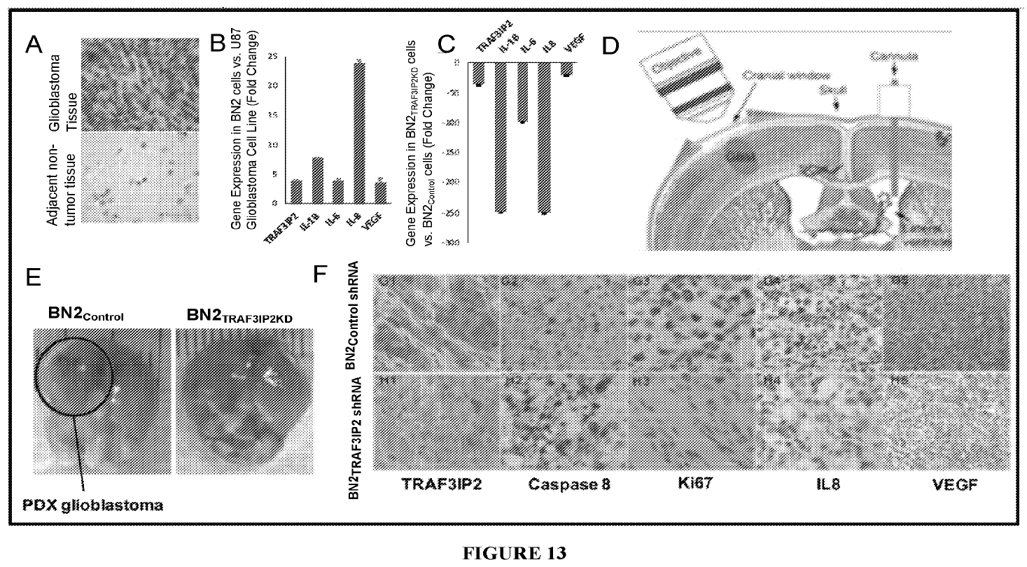

To explore the role of TRAF3IP2 in glioblastoma tumorigenesis, the following was performed, and the results are shown in FIG. 13. Histology on primary glioblastoma tissue (BN2) shows high expression of TRAF3IP2 compared to adjacent nontumor tissue. Sections were counterstained with hematoxylin (blue). Scale bar 100 mm (A). mRNA analysis indicates higher expression of TRAF3IP2, proinflammatory, and angiogenesis genes compared to U87 cells (B). Silencing TRAF3IP2 in BN2 cells suppresses the expression of TRAF3IP2 proinflammatory, and angiogenesis markers compared to BN2Control shRNA (C). Schema of the experimental procedure used for establishing glioblastoma brain model in left SSCx (D). Administration of BN2TRAF3IP2 KD (3×105 cells in PBS and Martigel) were injected into the injected into the left SSCx of NSG mice (n=9/group). Due to significant neurologic symptoms including, neuronal dysfunction, grip strength test, and deficit in the rotarod tasks, (in addition to weight loss, and blindness) in the animals injected with BN2Control shRNA, they were euthanized 28 days post-injection. The experiment was terminated on day 72 for the BN2T_{RAF3IP2 KD} injected animals. A significantly larger tumor was observed in BN2Control shRNA tumor on day 28 post tumor induction compare to the BN2_{TRAF3IP2} KD injected mice on day 72 on the tumor induction (E). Photomicrographs of histology of BN2_{TRAF3IP2 KD} (bottom panel) and BN2Control shRNA tumors (top panel) immunohistochemical detection of TRAF3IP2, Ki67, IL-8, and VEGF in these tumors, analyzed on day 28 (for BN2Control shRNA injected mice) and day 72 (for BN2_{TRAF3IP2 KD} injected mice) posttumor induction. Scale bar, 20 μm (F).

### Silencing TRAF3IP2 Inhibits Neoangiogenesis by Inhibiting HIF1-αExpression

Our data show that targeting TRAF3IP2 suppresses the pro-angiogenic NF-κB and mTOR, and inhibits angiogenic processes in glioblastoma31. Our in vivo data indicate that treating glioblastoma PDX with TRAF3IP2-AO results in reduced tumor growth. Immunohistochemical analysis of the vessel structure within the TRAF3IP2-AO treated tumors revealed lower blood vessel density/angiogenesis in TRAF3IP2-AO treated tumors, indicated by reduced CD31 staining (an epithelial marker) compared to SCRAO treated tumors. In addition, staining for human-specific mitochondria showed significantly less number in glioblastoma cells (human cells) in residual TRAF3IP2-AO treated tumor tissue. This demonstrates a critical role for TRAF3IP2 in neoangiogenesis.

Also, it is notable that TRAF3IP2 induces the expression and release of pro-angiogenic mediators into the TME, activating endothelial cells to promote neo-vascularization.

### Silencing TRAF3IP2 Suppresses Tumor Growth in PDX Model

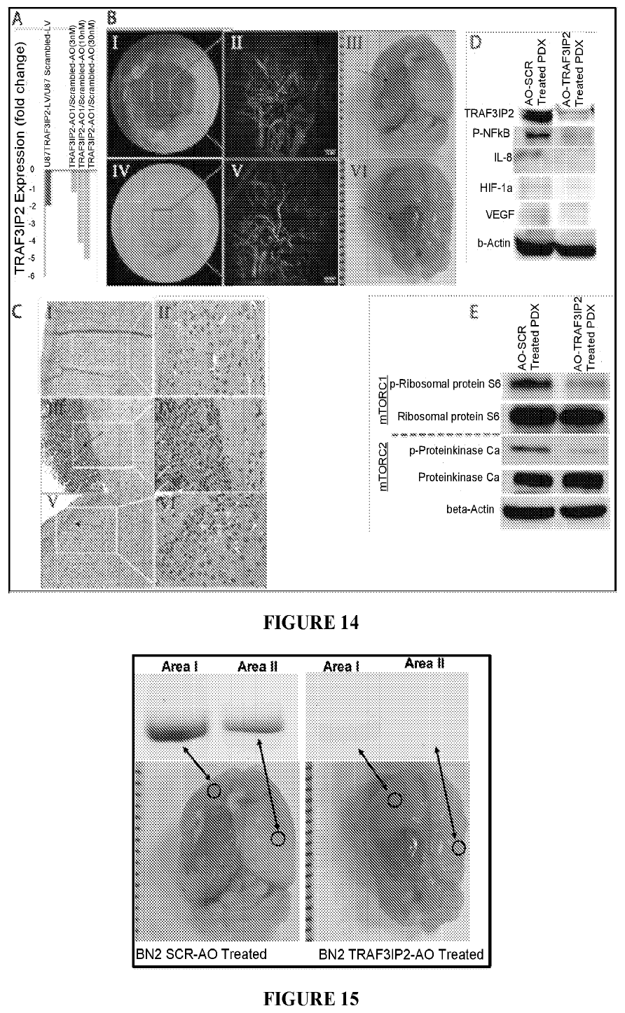

For the experiments reported here, the BN2 glioblastoma specimen was used in xenograft studies, and the results are shown in FIG. 14. Glioblastoma was induced by intracranial injection of tumor cells (into the left SSCx of NSG mice; 3×105 cells in PBS+Martigel; n=5 mice/group) isolated from primary glioblastoma tissue. At the time of the injection, a chronic cranial window was installed on the left side of the animal's skull that allowed in vivo 2PE imaging of tumor evolution.

For intracerebroventricular (ICV) administration of AO targeting TRAF3IP2 (TRAF3IP2-AO) or control scrambled AO (SCR-AO), a brain cannula was stereotaonctically implanted into the contralateral (right) ventricle (FIG. 14-A). Treatment of mice started on day 10 post-tumor induction with 5 μl every 48 h; 30 nanomolar AO (FIG. 14-B). The results indicate a marked reduction in tumor size following administration of TRAF3IP2-AO (vs. SCR-AO) (FIG. 14-BIII & BVI).

Using in vivo 2PE microscopy, angiograms of microvessels in glioblastoma tumors were captured through a cranial window after i.v. injection of fluorescein isothiocyanatedextran for visualization of blood vessels (green signal). Images correspond to day 24 post-tumor induction, after 8 ICV treatments with either SCR-AO (B, I&II; control group), or TRAF3IP2-AO (B IV&V). As demonstrated in FIG. 10B III&VI, tumors treated with TRAF3IP2-AO (FIG. 14B VI) showed significantly less growth and smaller volumes and displayed less vascularization compared to control scrambled (SCR)-AO treated mice (p<0.01) (FIG. 14-B III), demonstrating the efficacy of targeting and silencing TRAF3IP2 in suppressing tumor growth.

Protein expression in residual tumors confirmed that targeting TRAF3IP2 suppresses inflammation, neoangiogenesis, cell cycle and apoptosis (FIG. 14-D). Post-hoc histological analysis of the brain sections of TRAF3IP2-AO treated animals showed remarkable changes in tumor tissue compared to animals treated with SCR-AO (FIG. 14-CIII &CIV). The comparison of tissue sections with normal mouse brain shows a significantly smaller number of infiltrating cells in subcortical areas in TRAF3IP2-AO treated animals, while showing accumulation of pyknotic and apoptotic cells. Together, these histological and molecular data confirm the crucial role of TRAF3IP2 in malignant metabolism, inflammation and angiogenesis, and strongly suggest the mechanistic role of TRAF3IP2 in regulating multiple pro-tumorigenic pathways.

### Silencing TRAF3IP2 in Glioblastoma Prevents Intracranial Dissemination

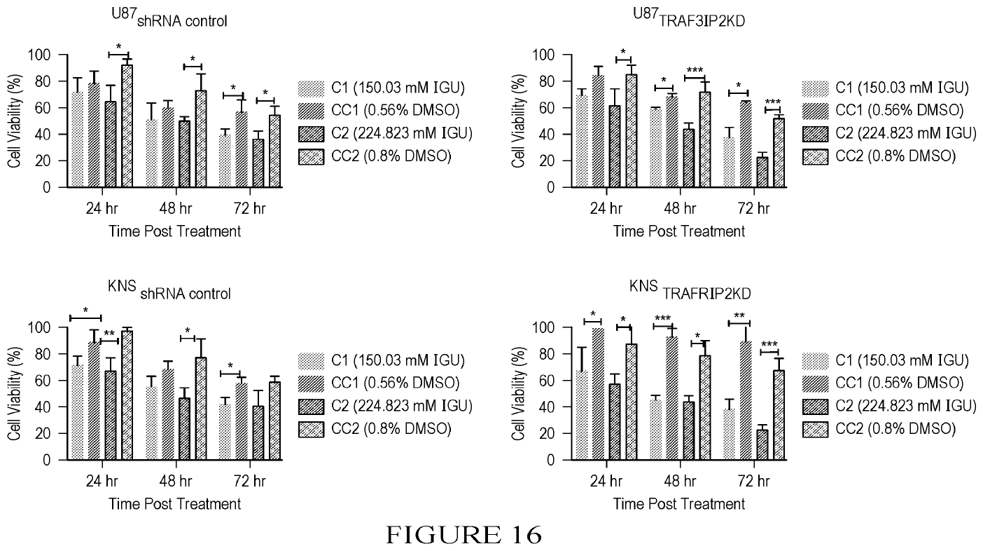

To detect intracranial micrometastasis, the animals in experiments detailed in FIG. 14 were biopsied from adjacent sections of brain. The biopsy specimen were subjected to PCR assay using primers that specifically detect human-specific a-satellite DNA sequence of the centromere region of chromosome 17. Areas evaluated include the left parietal lobe, at a distance of 2 mm from the focus of initial tumor inoculation (Area I) and the right SSCx equidistant from the interhemispheric fissure relative to the focus of initial tumor inoculation (Area II) (FIG. 15).

Results demonstrate markedly reduced metastasis at both sites in the TRAF3IP2-AO treated group as compared to control SCR-AO treated group (FIG. 15), demonstrating the role of TRAF3IP2 in metastasis and dissemination.

### Antisense Oilgonucleotide

Oligonucleotides are unmodified or chemically modified single-stranded DNA molecules. In general, they are relatively short (13-25 nucleotides) and hybridize (at least in theory) to a unique sequence in cells. Anti-sense oligonucleotides (ASOs) are single strands of DNA or RNA that are complementary to a chosen sequence. In the case of antisense RNA they prevent protein translation of certain messenger RNA strands by binding to them. If binding takes place, this DNA/RNA hybrid can be degraded by the enzyme RNase H. While the oligonucleotide may be susceptible to rapid degradation by nucleases, a 2′-methoxyethyl (2′-MOE) modified or 2′-O-methyl (2′-OMe) modified ASO is resistant to nucleases and has enhanced target binding and pharmacokinetics comparing to DNA. Therefore, the ASOs employed herein can be 2′-MOE or 2′-OMe modified or unmodified.

The inventors therefore investigated the possibility of silencing TRAF3IP2 with oligonucleotides and especially with more degradation-resistant oligonucleotides, as well as the suitable target binding sites within the gene.

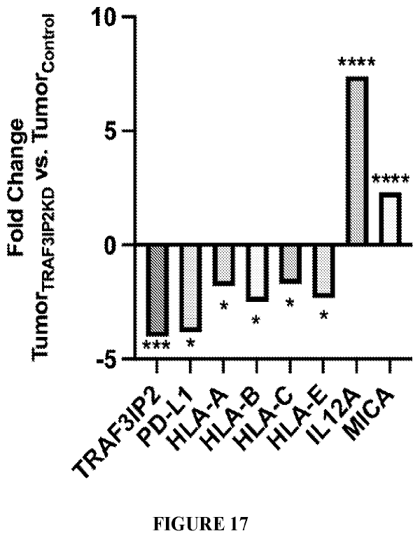

Several variants of NM_147200.2 (SEQ ID NO. 10) were investigated, including XM_011535386.2 (SEQ ID NO. 11), NM_147686.3 (SEQ ID NO. 12), NM_001164281.2 (SEQ ID NO. 9), XM006715319.4 (SEQ ID NO. 13), and NM_001164283.2 (SEQ ID NO. 14). Highly conserved regions appear in these sequences, as shown in FIG. 17A, suggesting that target binding sequence may be available within the conserved regions. TRAF3IP2 gene has 10 exons, among which exons 9 and 10 are conserved across the known variants. By targeting the most conserved regions across all variants, these oligonucleotides are believed to be able to block most of the TRAF3IP2 activity. Therefore, anti-sense oligonucleotides (ASOs) can be designed to specifically target these exons to obtain more universal applicability.

Experiments can also be designed to test the silencing effect of the ASOs across different cell lines from different tissues, in order to validate their efficacy on the TRAF3IP2 variants. It is expected that similar silencing of TRAF3IP2 expression can be achieved because these ASOs are designed to target only the conserved regions across known variants.

It is also contemplated that personalized silencing can be achieved, as the cost of producing the silencing sequences will continue to decline. As such, the need to target only the conserved regions across TRAF3IP2 variants may be obviated. Certain regions within TRAF3IP2 may be even more susceptible to silencing on an individualized basis, and the inventive concept described herein can be readily applied.

### Determining Malignancy of Tumor

TRAF3IP2 has different variants due to its splicing pattern. The inventors discovered certain unspliced TRAF3IP2 variants are more frequently present in cancer cells, particularly breast cancer and glioblastoma. The differentiation between non-malignant somatic cells and malignant cells is based on the cellular quotient of unspliced (very rare in normal cells) and the amount of spliced TRAF3IP2 expression, which is significantly greater in malignant cells.

As initially with a growing tumor, there are little or no tumor cells outside of a beginning and growing tumor to be found in circulation. To be able to detect these early tumor cells would be beneficial in early cancer detection. The detection is based on the fact that even tiny tumors develop a central necrosis that is cleaned up by macrophages. These macrophages go back in circulation and can be found and isolated from blood, where the macrophages migrate to the spleen discharge the dead tumor cells that still contain the genetic materials of the tumor. Therefore, by identifying the unspliced TRAF3IP2 gene, one can confirm that a malignant event is ongoing inside the patient. Further genetic analysis of the tumor cell material can yield indication as to the type and location of the tumor.

For example, SEQ ID NO. 3-4 are found in malignant tumor cells. (SEQ ID NO. 3: NP_671733.2; SEQ ID NO. 4: NM_147200.3; SEQ ID NO. 5: NP_679211.2; SEQ ID NO. 6: NM_147686.4) It is therefore possible to determine the malignancy of a tumor by detecting the presence of at least a portion of SEQ ID NO. 3-4. More specifically, the presence of the first nine amino acids in SEQ ID NO. 3 (mppqlqetr, SEQ ID NO. 7), as well as bp 196-222 in SEQ ID NO. 4 (atgcc tectcagctt caagaaacta ga, SEQ ID NO. 8) are of particular interest, and can be used as the basis for detection.

The first step is to collect a biological sample from a subject having tumor, such as blood, bodily fluids, tumor tissue, etc. Then the biological sample is tested for the presence for SEQ ID NO. 7 or 8 to determine whether the tumor is malignant. This way it is possible to detect and determine the presence of malignant tumor cells in the subject before the tumor is discovered.

Optionally, it is possible to isolate the macrophages from the biological sample by targeting the macrophage-specific cell surface markers, such as CD14, CD16, CD64, CD68, Cd71 and CCR5. Other markers known to a person skilled in the art can also be used without deviating from the spirit of this disclosure. An antibody specifically binding to SEQ ID NO. 7 can be used for detecting the presence of the peptide in the sample. Alternatively, qPCR or similar techniques can be used to detect the presence of SEQ ID NO. 8.

Once the amount of unspliced TRAF3IP2 in the sample is determined, a sample ratio of unspliced TRAF3IP2 to spliced TRAF3IP2 can be established. The sample ratio can then be compared to a baseline ratio previously established in normal somatic cells. A significant increase, such as an increase of 20%, 30%, 40%, 50% or more, would indicate that there is already malignant tumor cells inside the subject's body. Additional samples cam be collected to determine the type and location of the tumor cells to further facilitate early cancer detection.

The are other TRAF3IP2 variants that may be used to detect the malignant type of tumors, and their detection can also be used for early diagnosis and treatment.

### Targeting TRAF3IP2 Improves Anti-Tumor Immune Responses

The hallmark immunosuppressive microenvironment in breast cancer and GBM is characterized by decreased CD8 tumor infiltrating lymphocytes (TILs), decreased natural killer (NK) cell function, and increased immunosuppressive tumor associated macrophages (TAMs). These changes, the decreased immune response during aging, the rise in uncontrolled inflammation (SASP), and the increased risk of breast cancer and GBM in aging are critical barriers to the use of immunotherapy for treating these malignancies.

Current immunotherapy involves immune checkpoint inhibition. PD-L1 expressed on tumor cells inhibits the anti-tumor cytotoxic CD8 T cells; blockade of PD-L1 through a monoclonal antibody has shown some promise in improving the anti-tumor immune response, but it has been faced with immunotherapeutic resistance and lack of endogenous immune stimulation and response. Indeed, to achieve a functional cure, it is likely necessary to engage additional immune responses with greater potency.

The inventors showed that targeting TRAF3IP2 significantly reduces endogenous PD-L1 expression of tumor cells, which removes the possibility of the tumor cell endogenously enhancing PD-L1 expression due to a negative feedback mechanism from monoclonal antibody targeting PD-L1. This change will result in significantly increased anti-tumor CD8 T cell activity, and increased TILs in the microenvironment, and provide synergy with PD-L1 blocking drugs.

Next, it is demonstrated that targeting TRAF3IP2 will lead to enhanced NK cell mediated tumor killing via multiple mechanisms. First, targeting TRAF3IP2 significantly reduces expression of HLA-E on tumor cells. HLA-E is overexpressed in malignant cells and protects cancer cells from NK cell mediated destruction. For example the expression of HLA-E directly correlated with glioma grade(1). These make HLA-E an ideal target for improving tumor susceptibility to NK cell mediated tumor killing. Secondly, NK cells kill tumor cells through an antigen-independent manner, a scenario in which loss of MHC class 1 proteins causes loss of self-recognition by NK cells and triggers cytotoxic mediators to kill tumor cells(2). It was recently shown that a decrease of all MHC I class molecules (HLA-A, HLA-B, and HLA-C) is necessary to enable tumors to be susceptible to all NK cells, and to avoid development of tumor resistance to NK cell immunity(2).

It is shown that, referring to FIG. 17, targeting TRAF3IP2 in tumors downregulates all MHC I class molecules (HLA-A, HLA-B, and HLA-C), thus rendering tumors susceptible to NK cell killing. It has been confirmed that targeting TRAF3IP2 significantly reduces HLA-A, HLA-B, and HLA-C coordinately and consistently using immunohistochemistry (IHC) on tumors with TRAF3IP2 silenced vs control tumors.

Next, it is demonstrated that targeting TRAF3IP2 results in a significant upregulation of IL12A, a potent activator of both anti-tumor cytotoxic and T-helper cells as well as NK cells; therefore it is expected that this mechanisms would enhance ability of targeting TRAF3IP2 to enhance anti-tumor immunity. For example, intratumoral IL-12 increased efficacy of immunotherapy in GBM, and decreased the immunosuppressive microenvironment through decreasing inhibitor Tregs. MICA is a ligand expressed by cells and acts as a “kill me” signal for primarily NK cells through engagement of the NKG2D receptor. Tumor cells downregulate MICA for immune evasion and resistance to NK and cytotoxic T cell killing. Indeed, approaches to upregulate or prevent the downregulation of MICA have improved NK-mediated anti-tumor responses. The data demonstrates that targeting TRAF3IP2 significantly upregulates MICA, and will therefore enhance NK-cell mediated tumor killing.

Overall, these data suggest that targeting TRAF3IP2 can transform the immunosuppressive tumor microenvironment into an immunostimulatory microenvironment with effective tumor killing. Therefore, the TRAF3IP2-targeting composition can be used prior to, concurrently with, or after administration of cancer immunotherapeutic agent known to persons skilled in the art. Non-limiting examples of cancer immunotherapeutic agent include: alemtuzumab (monoclonalantibody against CD52), atezolizumab (MAb against PD-L1), aveluman (MAb against PD-L1), ipilimumab (MAb against CTLA4), elotuzumab (MAb against SLAMF7), ofatumumab (MAb against CD20), nivolumab (MAb against PD-1), pembrolizumab (Mab against PD-1), rituximab (Mab against CD20), and durvalumab (Mab against PD-L1).

### Therapeutic Significance of Targeting TRAF3IP2 in the Regression of Pre-Existing Glioblastoma and Breast Cancer

Based on the data described above, it is shown that TRAF3IP2 plays an important role in tumorigenesis, angiogenesis, metastasis and cancer cell metabolism, especially in breast cancer or gliobastoma as well as other possible cancers that overexpresses TRAF3IP2. The data also shows that targeting and silencing TRAF3IP2 in the cancer cells leads to suppression of tumor growth and reduces metastasis. Therefore, to prevent metastasis triggered by invasive procedures, it is proposed that a targeting/suppression/silencing step of TRAF3IP2 in the tumor can be performed prior to the invasive procedures to reduce or prevent metastasis.

The results show that treating existing tumors formed by the wild type U87 glioblastoma cells with TRAF3IP2 shRNA significantly reduces tumor size in the flank xenograft model.

The following reference are incorporated by reference herein in its entirety for all purposes:
1. Lombardi, G. et al. Diagnostic value of plasma and urinary 2-hydroxyglutarate to identify patients with isocitrate dehydrogenase-mutated glioma. Oncologist 20, 562-567, doi: 10.1634/theoncologist.2014-0266 (2015).
2 Ladomersky, E. et al. The Coincidence Between Increasing Age, Immunosuppression, and the Incidence of Patients With Glioblastoma. Front Pharmacol 10, 200, doi: 10.3389/fphar.2019.00200 (2019).
3 Arrigo, R. T., Boakye, M. & Skirboll, S. L. Patterns of care and survival for glioblastoma patients in the Veterans population. J Neurooncol 106, 627-635, doi:10.1007/s11060-011-0702-6 (2012).
4 Taylor, O. G., Brzozowski, J. S. & Skelding, K. A. Glioblastoma Multiforme: An Overview of Emerging Therapeutic Targets. Front Oncol 9, 963, doi:10.3389/fonc.2019.00963 (2019).
5 Wick, W. et al. Temozolomide chemotherapy alone versus radiotherapy alone for malignant astrocytoma in the elderly: the NOA-08 randomised, phase 3 trial. Lancet Oncol 13, 707-715, doi: 10.1016/S1470-2045(12)70164-X (2012).
6 Li, Y., Ali, S., Clarke, J. & Cha, S. Bevacizumab in Recurrent Glioma: Patterns of Treatment Failure and Implications. Brain Tumor Res Treat 5, 1-9, doi:10.14791/btrt.2017.5.1.1 (2017).
7 Louis, D. N. et al. The 2016 World Health Organization Classification of Tumors of the Central Nervous System: a summary. Acta Neuropathol 131, 803-820, doi: 10.1007/s00401-016-1545-1 (2016).
8 Nonoguchi, N. et al. TERT promoter mutations in primary and secondary glioblastomas. Acta Neuropathol 126, 931-937, doi: 10.1007/s00401-013-1163-0 (2013).
9 Ohgaki, H. & Kleihues, P. The definition of primary and secondary glioblastoma. Clin Cancer Res 19, 764-772, doi:10.1158/1078-0432.CCR-12-3002 (2013).
10 Colotta, F., Allavena, P., Sica, A., Garlanda, C. & Mantovani, A. Cancer-related inflammation, the seventh hallmark of cancer: links to genetic instability. Carcinogenesis 30, 1073-1081, doi: 10.1093/carcin/bgp127 (2009).
11 Sowers, J. L., Johnson, K. M., Conrad, C., Patterson, J. T. & Sowers, L. C. The role of inflammation in brain cancer. Adv Exp Med Biol 816, 75-105, doi: 10.1007/978-3-0348-0837-8_4 (2014).
12 Quail, D. F. & Joyce, J. A. The Microenvironmental Landscape of Brain Tumors. Cancer Cell 31, 326-341, doi: 10.1016/j.ccell.2017.02.009 (2017).
13 Persano, L., Rampazzo, E., Basso, G. & Viola, G. Glioblastoma cancer stem cells: role of the microenvironment and therapeutic targeting. Biochem Pharmacol 85, 612-622, doi:10.1016/j.bcp.2012.10.001 (2013).
14 Reinhard, J., Brosicke, N., Theocharidis, U. & Faissner, A. The extracellular matrix niche microenvironment of neural and cancer stem cells in the brain. Int J Biochem Cell Biol 81, 174-183, doi: 10.1016/j.biocel.2016.05.002 (2016).
15 Yi, Y., Hsieh, I. Y., Huang, X., Li, J. & Zhao, W. Glioblastoma Stem-Like Cells: Characteristics, Microenvironment, and Therapy. Front Pharmacol 7, 477, doi:10.3389/fphar.2016.00477 (2016).
16 Alt, E. U. et al. TRAF3IP2, a novel therapeutic target in glioblastoma multiforme. Oncotarget 9, 29772-29788, doi:10.18632/oncotarget.25710 (2018).
17 Alt, E. U. et al. Targeting TRAF3IP2, Compared to Rab27, is More Effective in Suppressing the Development and Metastasis of Breast Cancer. Sci Rep 10, 8834, doi: 10.1038/s41598-020-64781-z (2020).
18 Matossian, M. D. et al. Drug resistance profiling of a new triple negative breast cancer patient-derived xenograft model. BMC Cancer 19, 205, doi: 10.1186/s12885-019-5401-2 (2019).
19 Matossian, M. D. et al. A novel patient-derived xenograft model for claudin-low triple-negative breast cancer. Breast Cancer Res Treat 169, 381-390, doi:10.1007/s10549-018-4685-2 (2018).
20 Matossian, M. D. et al. Panobinostat suppresses the mesenchymal phenotype in a novel claudin-low triple negative patientderived breast cancer model. Oncoscience 5, 99-108, doi: 10.18632/oncoscience.412 (2018).
21 Kiseleva, L. N., Kartashev, A. V., Vartanyan, N. L., Pinevich, A. A. & Samoilovich, M. P. Characteristics of A172 and T98g Cell Lines. Tsitologiia 58, 349-355 (2016).
22 Becker, M. et al. Sensitive PCR method for the detection and real-time quantification of human cells in xenotransplantation systems. Br J Cancer 87, 1328-1335, doi:10.1038/sj.bjc.6600573 (2002).
23 Muller, F. The nature and mechanism of superoxide production by the electron transport chain: Its relevance to aging. J Am Aging Assoc 23, 227-253, doi:10.1007/s11357-000-0022-9 (2000).
24 Han, D., Williams, E. & Cadenas, E. Mitochondrial respiratory chain-dependent generation of superoxide anion and its release into the intermembrane space. Biochem J 353, 411-416 (2001).
25 Buchholz, M. et al. Innovative substance 2250 as a highly promising anti-neoplastic agent in malignant pancreatic carcinoma—in vitro and in vivo. BMC Cancer 17, 216, doi: 10.1186/s12885-017-3204-x (2017).
26 Weydert, C. J. & Cullen, J. J. Measurement of superoxide dismutase, catalase and glutathione peroxidase in cultured cells and tissue. Nat Protoc 5, 51-66, doi:10.1038/nprot.2009.197 (2010).
27 Magnuson, B., Ekim, B. & Fingar, D. C. Regulation and function of ribosomal protein S6 kinase (S6K) within mTOR signaling networks. Biochem J 441, 1-21, doi: 10.1042/BJ20110892 (2012).
28 Xue, X. et al. Protein kinase Calpha drives fibroblast activation and kidney fibrosis by stimulating autophagic flux. J Biol Chem 293, 11119-11130, doi: 10.1074/jbc.RA118.002191 (2018).
29 Ribatti, D. et al. Angiogenesis and Antiangiogenesis in Triple-Negative Breast cancer. Transl Oncol 9, 453-457, doi: 10.1016/j.tranon.2016.07.002 (2016).
30 Dodd, K. M., Yang, J., Shen, M. H., Sampson, J. R. & Tee, A. R. mTORC1 drives HIF-1alpha and VEGF-A signalling via multiple mechanisms involving 4E-BP1, S6K1 and STAT3. Oncogene 34, 2239-2250, doi:10.1038/onc.2014.164 (2015).
31 Xie, T. X., Xia, Z., Zhang, N., Gong, W. & Huang, S. Constitutive NF-kappaB activity regulates the expression of VEGF and IL-8 and tumor angiogenesis of human glioblastoma. Oncol Rep 23, 725-732 (2010).
32 Wang, C. et al. Oestrogen Inhibits VEGF Expression And Angiogenesis In Triple-Negative Breast Cancer By Activating GPER-1. J Cancer 9, 3802-3811, doi:10.7150/jca.29233 (2018).
33 Friedmann-Morvinski, D. et al. Targeting NF-kappaB in glioblastoma: A therapeutic approach. Sci Adv 2, e1501292, doi: 10.1126/sciadv.1501292 (2016).
34 Cahill, K. E., Morshed, R. A. & Yamini, B. Nuclear factor-kappaB in glioblastoma: insights into regulators and targeted therapy. Neuro Oncol 18, 329-339, doi:10.1093/neuonc/nov265 (2016).
35 Robe, P. A. et al. Early termination of ISRCTN45828668, a phase 1/2 prospective, randomized study of sulfasalazine for the treatment of progressing malignant gliomas in adults. BMC Cancer 9, 372, doi: 10.1186/1471-2407-9-372 (2009).
36 Sennino, B. & McDonald, D. M. Controlling escape from angiogenesis inhibitors. Nat Rev Cancer 12, 699-709, doi: 10.1038/nrc3366 (2012).
37 Emdad, L. et al. AEG-1/MTDH/LYRIC: signaling pathways, downstream genes, interacting proteins, and regulation of tumor angiogenesis. Adv Cancer Res 120, 75-111, doi: 10.1016/B978-0-12-401676-7.00003-6 (2013).
38 Ning, Y. et al. Interleukin-8 is associated with proliferation, migration, angiogenesis and chemosensitivity in vitro and in vivo in colon cancer cell line models. Int J Cancer 128, 2038-2049, doi: 10.1002/ijc.25562 (2011).
39 Levasseur, J. E., Wei, E. P., Raper, A. J., Kontos, A. A. & Patterson, J. L. Detailed description of a cranial window technique for acute and chronic experiments. Stroke 6, 308-317 (1975).
40 Ito, M. et al. NOD/SCID/gamma(c)(null) mouse: an excellent recipient mouse model for engraftment of human cells. Blood 100, 3175-3182, doi: 10.1182/blood-2001-12-0207 (2002).
41. Wischhusen, J., Friese, M. A., Mittelbronn, M., Meyermann, R., and Weller, M. (2005) HLA-E protects glioma cells from NKG2D-mediated immune responses in vitro: implications for immune escape in vivo. J Neuropathol Exp Neurol 64, 523-528
42. Maskalenko, N. A., Zhigarev, D., and Campbell, K. S. (2022) Harnessing natural killer cells for cancer immunotherapy: dispatching the first responders. Nat Rev Drug Discov
43. Agliardi, G., Liuzzi, A. R., Hotblack, A., De Feo, D., Nunez, N., Stowe, C. L., Friebel, E., Nannini, F., Rindlisbacher, L., Roberts, T. A., Ramasawmy, R., Williams, I. P., Siow, B. M., Lythgoe, M. F., Kalber, T. L., Quezada, S. A., Pule, M. A., Tugues, S., Straathof, K., and Becher, B. (2021) Intratumoral IL-12 delivery empowers CAR-T cell immunotherapy in a pre-clinical model of glioblastoma. Nat Commun 12, 444
44. Ferrari de Andrade, L., Tay, R. E., Pan, D., Luoma, A. M., Ito, Y., Badrinath, S., Tsoucas, D., Franz, B., May, K. F., Jr., Harvey, C. J., Kobold, S., Pyrdol, J. W., Yoon, C., Yuan, G. C., Hodi, F. S., Dranoff, G., and Wucherpfennig, K. W. (2018) Antibody-mediated inhibition of MICA and MICB shedding promotes NK cell-driven tumor immunity. Science 359, 1537-1542

What is claimed is:

1. A method of preventing surgery-, diagnostic- or intervention-related micrometastasis on a subject having cancer, wherein the cancer cells have increased TRAF3IP2 expression in comparison with a non-cancer cell, said method comprising the steps of:
a) administering a pharmaceutical composition into the subject, and
b) performing (i) surgery to treat the cancer, (ii) diagnostic for the cancer, or (iii) intervention to treat the cancer;
c) wherein the pharmaceutical composition comprises at least one targeting sequence for TRAF3IP2 in a pharmaceutically acceptable carrier in an amount effective for the prevention of micrometastasis.

2. The method of claim 1, wherein the cancer is breast cancer or glioblastoma multiforme.

3. The method of claim 1, wherein the targeting sequence for TRAF3IP2 is selected from the group consisting of: siRNA, a miRNA, a shRNA, an antisense RNA, or an antisense oligonucleotide.

4. The method of claim 3, wherein said composition comprises an expression vector encoding the targeting sequence for TRAF3IP2 operably coupled to an inducible promoter.

5. The method of claim 1, wherein the targeting sequence for TRAF3IP2 has the following sequence:
(SEQ ID NO: 1)
CCGGCATGGAACTATCATTACCATTCTCGAGAATGGTAATGATAGTTCCA
TGTTTTTT.

6. The method of claim 1, wherein the targeting sequence for TRAF3IP2 is an anti-sense oligonucleotide having the sequence of:
(SEQ ID NO: 2)
mG*mG*mU*mG*mG*G*C*A*C*A*T*G*C*T*C*mC*mU*mU*mC*mU.

7. The method of claim 1, wherein the micrometastasis of the cancer is reduced by at least 50% as compared to the same treatment without administering the pharmaceutical composition.

8. The method of claim 1, wherein the administering step comprises parenteral administration, including injection into a tumor or its metastasis site by transcutaneous, intraarterial, intraductal, intravenous, intradermal, intramuscular, intraperitoneal, or subcutaneous administration.

9. The method of claim 1, wherein said targeting sequence reduces the expression of the TRAF3IP2 gene by at least 5-fold as compared to without the targeting sequence for TRAF3IP2.

10. A method of treating a patient with cancer, the cancer having increase in TRAF3IP2 expression as compared to a normal cell, said method comprising the steps of:
a) administering a pharmaceutical composition into a subject before, during or in connection with at least one of the following procedure: surgery, diagnostic or therapeutic intervention, chemotherapy, radiation therapy, immunotherapy or targeted intervention;
b) wherein the pharmaceutical composition comprises at least one targeting sequence for TRAF3IP2 in a pharmaceutically acceptable carrier in an amount effective for prevention of micrometastasis, wherein the targeting sequence suppresses expression of TRAF3IP2.

11. The method of claim 10, wherein the cancer is breast cancer or glioblastoma multiforme.

12. The method of claim 10, wherein the targeting sequence for TRAF3IP2 is selected from the group consisting of: siRNA, miRNA, shRNA, an antisense RNA, or of an antisense oligonucleotide.

13. (canceled)

14. The method of claim 10, wherein the silencing sequence for TRAF3IP2 is a SEQ ID NOs. 1 or 2.

15. The method of claim 10, wherein the procedure in step (a) is chemotherapy, and wherein a chemotherapy agent is used, and wherein the chemotherapy agent is selected from the group consisting of: Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vemurafenib, Vinblastine, Vincristine, and Vindesine.

16. (canceled)

17. (canceled)

18. The method of claim 10, wherein mTOR activation in the cancer cell, the NF-κB activity in the cancer cells, the tumor growth, or angiogenesis of the cancer is inhibited.

19. (canceled)

20. (canceled)

21. (canceled)

22. (canceled)

23. (canceled)

24. (canceled)

25. (canceled)

26. (canceled)

27. A method of determining malignancy of a tumor in a patient, comprising the steps of:
a) obtaining a biological sample from a patient; and
b) detecting presence of a unspliced TRAF3IP2 in the biological sample;
wherein the presence of the unspliced TRAF3IP2 in the biological sample indicates malignancy of the tumor.

28. The method of claim 27, wherein in step a) the unspliced TRAF3IP2 is detecting presence of a portion of SEQ ID NO: 3 or 4 in the biological sample.

29. The method of claim 27, wherein in step b) detects the presence of SEQ ID NO. 7 or 8.

30. (canceled)

31. The method of claim 27, further comprising the step of a-1) prior to step b):
a-1) isolating macrophages from the biological sample.

32. The method of claim 27, further comprising the step of:
c) calculating a ratio of unspliced TRAF3IP2 to spliced TRAF3IP2.

33. The method of claim 27, wherein the tumor is pancreatic cancer, lung cancer, breast cancer, ovarian cancer, prostate cancer, glioblastoma, liver gastric cancer, bone cancer.

34. (canceled)

35. (canceled)


SEQUENCE LISTING (14 sequences)

SEQ ID NO: 1 - DNA, 58 bp, Homo sapiens
ccggcatggaactatcattaccattctcgagaatggtaatgatagttccatgtttttt

SEQ ID NO: 2 - DNA, 30 bp, Homo sapiens
gmgmumgmgmgcacatgctccmumumcmum

SEQ ID NO: 3 - protein, 574 aa, Homo sapiens
MPPQLQETRMNRSIPVEVDESEPYPSQLLKPIPEYSPEEESEPPAPNIRNMAPNSLSAPTMLHNSSGDFSQAHSTLKLANHQRPVSRQVTCLRTQVLEDSEDSFCRRHPGLGKAFPSGCSAVSEPASESVVGALPAEHQFSFMEKRNQWLVSQLSAASPDTGHDSDKSDQSLPNASADSLGGSQEMVQRPQPHRNRAGLDLPTIDTGYDSQPQDVLGIRQLERPLPLTSVCYPQDLPRPLRSREFPQFEPQRYPACAQMLPPNLSPHAPWNYHYHCPGSPDHQVPYGHDYPRAAYQQVIQPALPGQPLPGASVRGLHPVQKVILNYPSPWDHEERPAQRDCSFPGLPRHQDQPHHQPPNRAGAPGESLECPAELRPQVPQPPSPAAVPRPPSNPPARGTLKTSNLPEELRKVFITYSMDTAMEVVKFVNFLLVNGFQTAIDIFEDRIRGIDIIKWMERYLRDKTVMIIVAISPKYKQDVEGAESQLDEDEHGLHTKYIHRMMQIEFIKQGSMNFRFIPVLFPNAKKEHVPTWLQNTHVYSWPKNKKNILLRLLREEEYVAPPRGPLPTLQVVPL

SEQ ID NO: 4 - DNA, 5944 bp, Homo sapiens
gttcttctacttacctgggcccggagaaggtggagggagacgagaagccgccgagagccgactaccctccgggcccagtctgtctgtccgtggtggatctaagcctcatctgtatcctcttgtgatggcgtgaaggaaagccatggcagatttccagcctggtgatgctgtacagaacacaggtggcctgcttccatgcctcctcagcttcaagaaactagaatgaaccgaagcattcctgtggaggttgatgaatcagaaccatacccaagtcagttgctgaaaccaatcccagaatattccccggaagaggaatcagaaccacctgctccaaatataaggaacatggcacccaacagcttgtctgcacccacaatgcttcacaattcctccggagacttttctcaagctcactcaaccctgaaacttgcaaatcaccagcggcctgtatcccggcaggtcacctgcctgcgcactcaagttctggaggacagtgaagacagtttctgcaggagacacccaggcctgggcaaagctttcccttctgggtgctctgcagtcagcgagcctgcgtctgagtctgtggttggagccctccctgcagagcatcagttttcatttatggaaaaacgtaatcaatggctggtatctcagctttcagcggcttctcctgacactggccatgactcagacaaatcagaccaaagtttacctaatgcctcagcagactccttgggcggtagccaggagatggtgcaacggccccagcctcacaggaaccgagcaggcctggatctgccaaccatagacacgggatatgattcccagccccaggatgtcctgggcatcaggcagctggaaaggcccctgcccctcacctccgtgtgttacccccaggacctccccagacctctcaggtccagggagttccctcagtttgaacctcagaggtatccagcatgtgcacagatgctgcctcccaatctttccccacatgctccatggaactatcattaccattgtcctggaagtcccgatcaccaggtgccatatggccatgactaccctcgagcagcctaccagcaagtgatccagccggctctgcctgggcagcccctgcctggagccagtgtgagaggcctgcaccctgtgcagaaggttatcctgaattatcccagcccctgggaccacgaagagaggcccgcacagagagactgctcctttccggggcttccaaggcaccaggaccagccacatcaccagccacctaatagagctggtgctcctggggagtccttggagtgccctgcagagctgagaccacaggttccccagcctccgtccccagctgctgtgcctagaccccctagcaaccctccagccagaggaactctaaaaacaagcaatttgccagaagaattgcggaaagtctttatcacttattcgatggacacagctatggaggtggtgaaattcgtgaactttttgttggtaaatggcttccaaactgcaattgacatatttgaggatagaatccgaggcattgatatcattaaatggatggagcgctaccttagggataagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctctgcccacccttcaggtggttcccttgtgacaccgttcatccccagatcactgaggccaggccatgtttggggccttgttctgacagcattctggctgaggctggtcggtagcactcctggctggtttttttctgttcctccccgagaggccctctggcccccaggaaacctgttgtgcagagctcttccccggagacctccacacaccctggctttgaagtggagtctgtgactgctctgcattctctgcttttaaaaaaaccattgcaggtgccagtgtcccatatgttcctcctgacagtttgatgtgtccattctgggcctctcagtgcttagcaagtagataatgtaagggatgtggcagcaaatggaaatgactacaaacactctcctatcaatcacttcaggctacttttatgagttagccagatgcttgtgtatcctcagaccaaactgattcatgtacaaataataaaatgtttactcttttgtaagattatgttttacttatctcaaaggagatacatataatttataatgatatgggcagttgcttccagggacatcaacaaagctgcttagatataatattagataaatataacagaccactctgtattaatggattaaagccagctagttaaacaaccctttttaaccataatcatggaagctttattcttgcaataaagatttttaggctgggcgcagtgactcacacctgtaatcccagcactttgggaagctaaggcaggcagatcatttgaggtcaggagtttgagaccagcctggccaacatggtgaaaccccatctctgctaaaattacaaaaaagttagccgggcatggtggtgtgcacctgtaatcccagctactcgggaggctgaggcaggagaatcacttgaacccgggaggcagaggttgcagtgagccgagatcatgtcactgcactctagcttgggagacagagcgagactccgtctcaaaaaacaaacaaacaaataaaaacacccatttttaacaaaacaactttatatagcatacagccatgattctaaatagtatgattatggttctcaggatctgactacataggtaaaaatatttgcatatgtgtatgaagtgttgggggatgtaggctagaattgtagtctgtgttctaattttggttctaccaccaattagctgtatgacctttagcaagtcctttaacttttcttagattccagggactcatttataaaatgacatggacaaaagcatctctaatcactctaaaagatttgaagtctaggacctaaattctaaatactcttttgaggagtgactgagttttcattttcataattatgtctctcagaggacaaatttacattttcttaacagagacattttcttcttctttttttttgtttgagacagagtctcgctctgtcgtccaggctggagtgcagtgctgcaatcttggctcactgcaacctgcgcctcctgggttcaagtgattcttctgcctcaacctcccaagtagctagacctataggcgcctgccaccatgcccagctaatttttgtatttttagtagagacagggtttcatattggccagactggtctcgaactcctgaccttgtgatccgcccacctcggcctcccaaagtgctgggattacaggtgtgagccaccacacccagccaacattttcctcttttaaaaaatatcttctcacgcctgtaatcccagcactttgggaggctgaggcaggcggatcatgaggtcaggagatcaagaccatcctggctaacacggtgaaactccatctctactaaaaatacaaaaaaaatagccgggcgtggtggcaggcgcctgtagtcccagctactggggaggctgaggcaggaaaatggtgtcaacccgggaggcggagcttgcagtgagccgagattgcgccactgcactccagcctgggcaatagagtgagactccgtctcaaaaaaaaaaaaaaaaaaaaaaacttcaacaataccctcaggttgataattttggatatctatctgtatctatatatcttgtttacctggtctccagaaaaagaacacatacacatatccatatataaaatatgtatacatgtatcaaatctacgtaaactataaaggtgggatggctttaattatggcccaagctactaagacaatgaagactttttggggctgcaagctactgcttcccttctttatctactagcctcttaaacaaggctcacttgtgctacaagacagtccaccgttttgttttttttttcttttttttgagacagggtctcactctttcccaggctgcagtacagtgacacagtctcagctcactgcagctttgaccttgccgggctcaggtgacccttacacttcagcctcccaagtagcagggactataggtgtgcaccaacatgcttggttaatttttgtattttttgtagagacagggttttgccatgttgtccaggctagtctcgaattcctgggctcaagtgattcacctgccttggcctcccaaagtgctaggattacagatgggagccaccacgcccagcccagtccagctcttatatgtagcacagggaaaggacaaatacttgtcaactataaataagaaacattgctaatgcattgcaaagaacactagtttcatttactttataacttagatgtctactgggtgagacgaatgtctttgttctttaaaaaataggaaaagagaagaaaaactagcataacataagtactcatttgtaagactttctgacatgtaacattagttccgtagttttgagacctggtagaactgactttcatatttggataacctggaaaacacccaaacacaaacttcaagtcttctttctcttttttcattatcttttttagtctgaggtgacaccatcattaaggattcgacacccgtttgtaaataaaatgacatcagcaattactctgaaatgtttctagtttgcaaagacttagcaatgtgatgttattaacccttcctcccttcagagacctgtcctaagctctgaaccactcattccttccactcttcttaccccaggtggttgatgagcagtggtccctggtgttccacaaagagtcattaaagtgttacagctggtagcactggtagcaaaaaaacaaaccaaaaagtacacacagacacacacacacacacgcacacatacacacacacacgcacttggccaagtgacaaaagcttggcccctgaaatttctatgagatccgatgaccaccaacatcaaagcatttttttttttttttttttgagacgtagtctcgctctgtcacccaggctagagtgcagtggtgcaatcacagctcactgcaacctccacctcccgggttcaagcgattctcctgcctcagcctctcgagtagctgtgactacaggcacctgccaccatgcccggctaattttttgtatttttagtagagacggggtttcaccgtgttagccaggatggtcttgatctcctgacctcgtgatccatccgcctcggcctcccaaattgctgggattacaggcatgagccaccacgcccggcccatcaaaggaattgtaacaactatttgagagcactgacaataagattaacactcggttgatttagatgttatgctggtcctcaggcattcatctttagatatttttggggtggaagtggggtagggctgacttagtaaaaataacctcttagcccaaaggctttattcagacttacaccgatttgaggggtgggtttgtggaatgcaaggttaggttcttacctaatatttgatgactaatttagaattttaaatgtaattttaaattttagtgactggtttcaaatctattttaacttctagattgttcaaagaggtctcagtacatggctacaatcaaagtattagactagctatttctcagctcagtgctcagaaaaattattactgttgatacctttttctttgtttcctgttaaataaatcacctctttaaagaca

SEQ ID NO: 5 - protein, 565 aa, Homo sapiens
MNRSIPVEVDESEPYPSQLLKPIPEYSPEEESEPPAPNIRNMAPNSLSAPTMLHNSSGDFSQAHSTLKLANHQRPVSRQVTCLRTQVLEDSEDSFCRRHPGLGKAFPSGCSAVSEPASESVVGALPAEHQFSFMEKRNQWLVSQLSAASPDTGHDSDKSDQSLPNASADSLGGSQEMVQRPQPHRNRAGLDLPTIDTGYDSQPQDVLGIRQLERPLPLTSVCYPQDLPRPLRSREFPQFEPQRYPACAQMLPPNLSPHAPWNYHYHCPGSPDHQVPYGHDYPRAAYQQVIQPALPGQPLPGASVRGLHPVQKVILNYPSPWDHEERPAQRDCSFPGLPRHQDQPHHQPPNRAGAPGESLECPAELRPQVPQPPSPAAVPRPPSNPPARGTLKTSNLPEELRKVFITYSMDTAMEVVKFVNFLLVNGFQTAIDIFEDRIRGIDIIKWMERYLRDKTVMIIVAISPKYKQDVEGAESQLDEDEHGLHTKYIHRMMQIEFIKQGSMNFRFIPVLFPNAKKEHVPTWLQNTHVYSWPKNKKNILLRLLREEEYVAPPRGPLPTLQVVPL

SEQ ID NO: 6 - DNA, 940 bp, Homo sapiens
gttcttctacttacctgggcccggagaaggtggagggagacgagaagccgccgagagccgactaccctccgggcccagtctgtctgtccgtggtggatctaagaaactagaatgaaccgaagcattcctgtggaggttgatgaatcagaaccatacccaagtcagttgctgaaaccaatcccagaatattccccggaagaggaatcagaaccacctgctccaaatataaggaacatggcacccaacagcttgtctgcacccacaatgcttcacaattcctccggagacttttctcaagctcactcaaccctgaaacttgcaaatcaccagcggcctgtatcccggcaggtcacctgcctgcgcactcaagttctggaggacagtgaagacagtttctgcaggagacacccaggcctgggcaaagctttcccttctgggtgctctgcagtcagcgagcctgcgtctgagtctgtggttggagccctccctgcagagcatcagttttcatttatggaaaaacgtaatcaatggctggtatctcagctttcagcggcttctcctgacactggccatgactcagacaaatcagaccaaagtttacctaatgcctcagcagactccttgggcggtagccaggagatggtgcaacggccccagcctcacaggaaccgagcaggcctggatctgccaaccatagacacgggatatgattcccagccccaggatgtcctgggcatcaggcagctggaaaggcccctgcccctcacctccgtgtgttacccccaggacctccccagacctctcaggtccagggagttccctcagtttgaacctcagaggtatccagcatgtgcacagatgctgcctcccaatctttccccacatgctccatggaactatcattaccattgtcctggaagtcccgatcaccaggtgccat

SEQ ID NO: 7 - protein, 9 aa, Homo sapiens
MPPQLQETR

SEQ ID NO: 8 - DNA, 27 bp, Homo sapiens
atgcctcctcagcttcaagaaactaga

SEQ ID NO: 9 - DNA, 6241 bp, Homo sapiens
aatgctgatgttttaagcagttagaggaggtggaagaagctcgactccctcttcttccccattatctgcccacaatcccctcctttggagctgctaatgattactaattcttaacattcgagttcaatctcctcccggagacaccctcccaggcgagggcactgcgactacactgaggttctgcccactcctgggcagcttcttagctgggtggcgaaaacaaaaatgccgcctaattggtcactggccctttctcatgaatgaaggaggtttctgttttaagaaataaagtgactcctcagccgttgattcactgcccacagggagattttgagcagaggcttcctaggctccgtagaaatttgcatacagcttccacttcctgcttcagagcctgttcttctacttacctgggcccggagaaggtggagggagacgagaagccgccgagagccgactaccctccgggcccagtctgtctgtccgtggtggatctaagaaactagaatgaaccgaagcattcctgtggaggttgatgaatcagaaccatacccaagtcagttgctgaaaccaatcccagaatattccccggaagaggaatcagaaccacctgctccaaatataaggaacatggcacccaacagcttgtctgcacccacaatgcttcacaattcctccggagacttttctcaagctcactcaaccctgaaacttgcaaatcaccagcggcctgtatcccggcaggtcacctgcctgcgcactcaagttctggaggacagtgaagacagtttctgcaggagacacccaggcctgggcaaagctttcccttctgggtgctctgcagtcagcgagcctgcgtctgagtctgtggttggagccctccctgcagagcatcagttttcatttatggaaaaacgtaatcaatggctggtatctcagctttcagcggcttctcctgacactggccatgactcagacaaatcagaccaaagtttacctaatgcctcagcagactccttgggcggtagccaggagatggtgcaacggccccagcctcacaggaaccgagcaggcctggatctgccaaccatagacacgggatatgattcccagccccaggatgtcctgggcatcaggcagctggaaaggcccctgcccctcacctccgtgtgttacccccaggacctccccagacctctcaggtccagggagttccctcagtttgaacctcagaggtatccagcatgtgcacagatgctgcctcccaatctttccccacatgctccatggaactatcattaccattgtcctggaagtcccgatcaccaggtgccatatggccatgactaccctcgagcagcctaccagcaagtgatccagccggctctgcctgggcagcccctgcctggagccagtgtgagaggcctgcaccctgtgcagaaggttatcctgaattatcccagcccctgggaccacgaagagaggcccgcacagagagactgctcctttccggggcttccaaggcaccaggaccagccacatcaccagccacctaatagagctggtgctcctggggagtccttggagtgccctgcagagctgagaccacaggttccccagcctccgtccccagctgctgtgcctagaccccctagcaaccctccagccagaggaactctaaaaacaagcaatttgccagaagaattgcggaaagtctttatcacttattcgatggacacagctatggaggtggtgaaattcgtgaactttttgttggtaaatggcttccaaactgcaattgacatatttgaggatagaatccgaggcattgatatcattaaatggatggagcgctaccttagggataccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctctgcccacccttcaggtggttcccttgtgacaccgttcatccccagatcactgaggccaggccatgtttggggccttgttctgacagcattctggctgaggctggtcggtagcactcctggctggtttttttctgttcctccccgagaggccctctggcccccaggaaacctgttgtgcagagctcttccccggagacctccacacaccctggctttgaagtggagtctgtgactgctctgcattctctgcttttaaaaaaaccattgcaggtgccagtgtcccatatgttcctcctgacagtttgatgtgtccattctgggcctctcagtgcttagcaagtagataatgtaagggatgtggcagcaaatggaaatgactacaaacactctcctatcaatcacttcaggctacttttatgagttagccagatgcttgtgtatcctcagaccaaactgattcatgtacaaataataaaatgtttactcttttgtaagattatgttttacttatctcaaaggagatacatataatttataatgatatgggcagttgcttccagggacatcaacaaagctgcttagatataatattagataaatataacagaccactctgtattaatggattaaagccagctagttaaacaaccctttttaaccataatcatggaagctttattcttgcaataaagatttttaggctgggcgcagtgactcacacctgtaatcccagcactttgggaagctaaggcaggcagatcatttgaggtcaggagtttgagaccagcctggccaacatggtgaaaccccatctctgctaaaattacaaaaaagttagccgggcatggtggtgtgcacctgtaatcccagctactcgggaggctgaggcaggagaatcacttgaacccgggaggcagaggttgcagtgagccgagatcatgtcactgcactctagcttgggagacagagcgagactccgtctcaaaaaacaaacaaacaaataaaaacacccatttttaacaaaacaactttatatagcatacagccatgattctaaatagtatgattatggttctcaggatctgactacataggtaaaaatatttgcatatgtgtatgaagtgttgggggatgtaggctagaattgtagtctgtgttctaattttggttctaccaccaattagctgtatgacctttagcaagtcctttaacttttcttagattccagggactcatttataaaatgacatggacaaaagcatctctaatcactctaaaagatttgaagtctaggacctaaattctaaatactcttttgaggagtgactgagttttcattttcataattatgtctctcagaggacaaatttacattttcttaacagagacattttcttcttctttttttttgtttgagacagagtctcgctctgtcgtccaggctggagtgcagtgctgcaatcttggctcactgcaacctgcgcctcctgggttcaagtgattcttctgcctcaacctcccaagtagctagacctataggcgcctgccaccatgcccagctaatttttgtatttttagtagagacagggtttcatattggccagactggtctcgaactcctgaccttgtgatccgcccacctcggcctcccaaagtgctgggattacaggtgtgagccaccacacccagccaacattttcctcttttaaaaaatatcttctcacgcctgtaatcccagcactttgggaggctgaggcaggcggatcatgaggtcaggagatcaagaccatcctggctaacacggtgaaactccatctctactaaaaatacaaaaaaaatagccgggcgtggtggcaggcgcctgtagtcccagctactggggaggctgaggcaggaaaatggtgtcaacccgggaggcggagcttgcagtgagccgagattgcgccactgcactccagcctgggcaatagagtgagactccgtctcaaaaaaaaaaaaaaaaaaaaaaacttcaacaataccctcaggttgataattttggatatctatctgtatctatatatcttgtttacctggtctccagaaaaagaacacatacacatatccatatataaaatatgtatacatgtatcaaatctacgtaaactataaaggtgggatggctttaattatggcccaagctactaagacaatgaagactttttggggctgcaagctactgcttcccttctttatctactagcctcttaaacaaggctcacttgtgctacaagacagtccaccgttttgttttttttttcttttttttgagacagggtctcactctttcccaggctgcagtacagtgacacagtctcagctcactgcagctttgaccttgccgggctcaggtgacccttacacttcagcctcccaagtagcagggactataggtgtgcaccaacatgcttggttaatttttgtattttttgtagagacagggttttgccatgttgtccaggctagtctcgaattcctgggctcaagtgattcacctgccttggcctcccaaagtgctaggattacagatgggagccaccacgcccagcccagtccagctcttatatgtagcacagggaaaggacaaatacttgtcaactataaataagaaacattgctaatgcattgcaaagaacactagtttcatttactttataacttagatgtctactgggtgagacgaatgtctttgttctttaaaaaataggaaaagagaagaaaaactagcataacataagtactcatttgtaagactttctgacatgtaacattagttccgtagttttgagacctggtagaactgactttcatatttggataacctggaaaacacccaaacacaaacttcaagtcttctttctcttttttcattatcttttttagtctgaggtgacaccatcattaaggattcgacacccgtttgtaaataaaatgacatcagcaattactctgaaatgtttctagtttgcaaagacttagcaatgtgatgttattaacccttcctcccttcagagacctgtcctaagctctgaaccactcattccttccactcttcttaccccaggtggttgatgagcagtggtccctggtgttccacaaagagtcattaaagtgttacagctggtagcactggtagcaaaaaaacaaaccaaaaagtacacacagacacacacacacacacgcacacatacacacacacacgcacttggccaagtgacaaaagcttggcccctgaaatttctatgagatccgatgaccaccaacatcaaagcatttttttttttttttttttgagacgtagtctcgctctgtcacccaggctagagtgcagtggtgcaatcacagctcactgcaacctccacctcccgggttcaagcgattctcctgcctcagcctctcgagtagctgtgactacaggcacctgccaccatgcccggctaattttttgtatttttagtagagacggggtttcaccgtgttagccaggatggtcttgatctcctgacctcgtgatccatccgcctcggcctcccaaattgctgggattacaggcatgagccaccacgcccggcccatcaaaggaattgtaacaactatttgagagcactgacaataagattaacactcggttgatttagatgttatgctggtcctcaggcattcatctttagatatttttggggtggaagtggggtagggctgacttagtaaaaataacctcttagcccaaaggctttattcagacttacaccgatttgaggggtgggtttgtggaatgcaaggttaggttcttacctaatatttgatgactaatttagaattttaaatgtaattttaaattttagtgactggtttcaaatctattttaacttctagattgttcaaagaggtctcagtacatggctacaatcaaagtattagactagctatttctcagctcagtgctcagaaaaattattactgttgatacctttttctttgtttcctgttaaataaatcacctctttaaagacagaaaaaaaaaaaaaa

SEQ ID NO: 10 - DNA, 2474 bp, Homo sapiens
ggagattttgagcagaggcttcctaggctccgtagaaatttgcatacagcttccacttcctgcttcagagcctgttcttctacttacctgggcccggagaaggtggagggagacgagaagccgccgagagccgactaccctccgggcccagtctgtctgtccgtggtggatctaagcctcatctgtatcctcttgtgatggcgtgaaggaaagccatggcagatttccagcctggtgatgctgtacagaacacaggtggcctgcttccatgcctcctcagcttcaagaaactagaatgaaccgaagcattcctgtggaggttgatgaatcagaaccatacccaagtcagttgctgaaaccaatcccagaatattccccggaagaggaatcagaaccacctgctccaaatataaggaacatggcacccaacagcttgtctgcacccacaatgcttcacaattcctccggagacttttctcaagctcactcaaccctgaaacttgcaaatcaccagcggcctgtatcccggcaggtcacctgcctgcgcactcaagttctggaggacagtgaagacagtttctgcaggagacacccaggcctgggcaaagctttcccttctgggtgctctgcagtcagcgagcctgcgtctgagtctgtggttggagccctccctgcagagcatcagttttcatttatggaaaaacgtaatcaatggctggtatctcagctttcagcggcttctcctgacactggccatgactcagacaaatcagaccaaagtttacctaatgcctcagcagactccttgggcggtagccaggagatggtgcaacggccccagcctcacaggaaccgagcaggcctggatctgccaaccatagacacgggatatgattcccagccccaggatgtcctgggcatcaggcagctggaaaggcccctgcccctcacctccgtgtgttacccccaggacctccccagacctctcaggtccagggagttccctcagtttgaacctcagaggtatccagcatgtgcacagatgctgcctcccaatctttccccacatgctccatggaactatcattaccattgtcctggaagtcccgatcaccaggtgccatatggccatgactaccctcgagcagcctaccagcaagtgatccagccggctctgcctgggcagcccctgcctggagccagtgtgagaggcctgcaccctgtgcagaaggttatcctgaattatcccagcccctgggaccacgaagagaggcccgcacagagagactgctcctttccggggcttccaaggcaccaggaccagccacatcaccagccacctaatagagctggtgctcctggggagtccttggagtgccctgcagagctgagaccacaggttccccagcctccgtccccagctgctgtgcctagaccccctagcaaccctccagccagaggaactctaaaaacaagcaatttgccagaagaattgcggaaagtctttatcacttattcgatggacacagctatggaggtggtgaaattcgtgaactttttgttggtaaatggcttccaaactgcaattgacatatttgaggatagaatccgaggcattgatatcattaaatggatggagcgctaccttagggataagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctctgcccacccttcaggtggttcccttgtgacaccgttcatccccagatcactgaggccaggccatgtttggggccttgttctgacagcattctggctgaggctggtcggtagcactcctggctggtttttttctgttcctccccgagaggccctctggcccccaggaaacctgttgtgcagagctcttccccggagacctccacacaccctggctttgaagtggagtctgtgactgctctgcattctctgcttttaaaaaaaccattgcaggtgccagtgtcccatatgttcctcctgacagtttgatgtgtccattctgggcctctcagtgcttagcaagtagataatgtaagggatgtggcagcaaatggaaatgactacaaacactctcctatcaatcacttcaggctacttttatgagttagccagatgcttgtgtatcctcagaccaaactgattcatgtacaaataataaaatgtttactcttttgtaaaaaaaaaaaaaaaaaa

SEQ ID NO: 11 - DNA, 2819 bp, Homo sapiens
tacaggcgtgagtcaccgcgctcggcaatgctgatgttttaagcagttagaggaggtggaagaagctcgactccctcttcttccccattatctgcccacaatcccctcctttggagctgctaatgattactaattcttaacattcgagttcaatctcctcccggagacaccctcccaggcgagggcactgcgactacactgaggttctgcccactcctgggcagcttcttagctgggtggcgaaaacaaaaatgccgcctaattggtcactggccctttctcatgaatgaaggaggtttctgttttaagaaataaagtgactcctcagccgttgattcactgcccacagggagattttgagcagaggcttcctaggctccgtagaaatttgcatacagcttccacttcctgcttcagagcctgttcttctacttacctgggcccggagaaggtggagggagacgagaagccgccgagagccgactaccctccgggcccagtctgtctgtccgtggtggatctaagatgcctctgcagcctcatctgtatcctcttgtgatggcgtgaaggaaagccatggcagatttccagcctggtgatgctgtacagaacacaggtggcctgcttccatgcctcctcagcttcaagaaactagaatgaaccgaagcattcctgtggaggttgatgaatcagaaccatacccaagtcagttgctgaaaccaatcccagaatattccccggaagaggaatcagaaccacctgctccaaatataaggaacatggcacccaacagcttgtctgcacccacaatgcttcacaattcctccggagacttttctcaagctcactcaaccctgaaacttgcaaatcaccagcggcctgtatcccggcaggtcacctgcctgcgcactcaagttctggaggacagtgaagacagtttctgcaggagacacccaggcctgggcaaagctttcccttctgggtgctctgcagtcagcgagcctgcgtctgagtctgtggttggagccctccctgcagagcatcagttttcatttatggaaaaacgtaatcaatggctggtatctcagctttcagcggcttctcctgacactggccatgactcagacaaatcagaccaaagtttacctaatgcctcagcagactccttgggcggtagccaggagatggtgcaacggccccagcctcacaggaaccgagcaggcctggatctgccaaccatagacacgggatatgattcccagccccaggatgtcctgggcatcaggcagctggaaaggcccctgcccctcacctccgtgtgttacccccaggacctccccagacctctcaggtccagggagttccctcagtttgaacctcagaggtatccagcatgtgcacagatgctgcctcccaatctttccccacatgctccatggaactatcattaccattgtcctggaagtcccgatcaccaggtgccatatggccatgactaccctcgagcagcctaccagcaagtgatccagccggctctgcctgggcagcccctgcctggagccagtgtgagaggcctgcaccctgtgcagaaggttatcctgaattatcccagcccctgggaccacgaagagaggcccgcacagagagactgctcctttccggggcttccaaggcaccaggaccagccacatcaccagccacctaatagagctggtgctcctggggagtccttggagtgccctgcagagctgagaccacaggttccccagcctccgtccccagctgctgtgcctagaccccctagcaaccctccagccagaggaactctaaaaacaagcaatttgccagaagaattgcggaaagtctttatcacttattcgatggacacagctatggaggtggtgaaattcgtgaactttttgttggtaaatggcttccaaactgcaattgacatatttgaggatagaatccgaggcattgatatcattaaatggatggagcgctaccttagggataagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctctgcccacccttcaggtggttcccttgtgacaccgttcatccccagatcactgaggccaggccatgtttggggccttgttctgacagcattctggctgaggctggtcggtagcactcctggctggtttttttctgttcctccccgagaggccctctggcccccaggaaacctgttgtgcagagctcttccccggagacctccacacaccctggctttgaagtggagtctgtgactgctctgcattctctgcttttaaaaaaaccattgcaggtgccagtgtcccatatgttcctcctgacagtttgatgtgtccattctgggcctctcagtgcttagcaagtagataatgtaagggatgtggcagcaaatggaaatgactacaaacactctcctatcaatcacttcaggctacttttatgagttagccagatgcttgtgtatcctcagaccaaactgattcatgtacaaataataaaatgtttactcttttgtaa

SEQ ID NO: 12 - DNA, 6244 bp, Homo sapiens
aatgctgatgttttaagcagttagaggaggtggaagaagctcgactccctcttcttccccattatctgcccacaatcccctcctttggagctgctaatgattactaattcttaacattcgagttcaatctcctcccggagacaccctcccaggcgagggcactgcgactacactgaggttctgcccactcctgggcagcttcttagctgggtggcgaaaacaaaaatgccgcctaattggtcactggccctttctcatgaatgaaggaggtttctgttttaagaaataaagtgactcctcagccgttgattcactgcccacagggagattttgagcagaggcttcctaggctccgtagaaatttgcatacagcttccacttcctgcttcagagcctgttcttctacttacctgggcccggagaaggtggagggagacgagaagccgccgagagccgactaccctccgggcccagtctgtctgtccgtggtggatctaagaaactagaatgaaccgaagcattcctgtggaggttgatgaatcagaaccatacccaagtcagttgctgaaaccaatcccagaatattccccggaagaggaatcagaaccacctgctccaaatataaggaacatggcacccaacagcttgtctgcacccacaatgcttcacaattcctccggagacttttctcaagctcactcaaccctgaaacttgcaaatcaccagcggcctgtatcccggcaggtcacctgcctgcgcactcaagttctggaggacagtgaagacagtttctgcaggagacacccaggcctgggcaaagctttcccttctgggtgctctgcagtcagcgagcctgcgtctgagtctgtggttggagccctccctgcagagcatcagttttcatttatggaaaaacgtaatcaatggctggtatctcagctttcagcggcttctcctgacactggccatgactcagacaaatcagaccaaagtttacctaatgcctcagcagactccttgggcggtagccaggagatggtgcaacggccccagcctcacaggaaccgagcaggcctggatctgccaaccatagacacgggatatgattcccagccccaggatgtcctgggcatcaggcagctggaaaggcccctgcccctcacctccgtgtgttacccccaggacctccccagacctctcaggtccagggagttccctcagtttgaacctcagaggtatccagcatgtgcacagatgctgcctcccaatctttccccacatgctccatggaactatcattaccattgtcctggaagtcccgatcaccaggtgccatatggccatgactaccctcgagcagcctaccagcaagtgatccagccggctctgcctgggcagcccctgcctggagccagtgtgagaggcctgcaccctgtgcagaaggttatcctgaattatcccagcccctgggaccacgaagagaggcccgcacagagagactgctcctttccggggcttccaaggcaccaggaccagccacatcaccagccacctaatagagctggtgctcctggggagtccttggagtgccctgcagagctgagaccacaggttccccagcctccgtccccagctgctgtgcctagaccccctagcaaccctccagccagaggaactctaaaaacaagcaatttgccagaagaattgcggaaagtctttatcacttattcgatggacacagctatggaggtggtgaaattcgtgaactttttgttggtaaatggcttccaaactgcaattgacatatttgaggatagaatccgaggcattgatatcattaaatggatggagcgctaccttagggataagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctctgcccacccttcaggtggttcccttgtgacaccgttcatccccagatcactgaggccaggccatgtttggggccttgttctgacagcattctggctgaggctggtcggtagcactcctggctggtttttttctgttcctccccgagaggccctctggcccccaggaaacctgttgtgcagagctcttccccggagacctccacacaccctggctttgaagtggagtctgtgactgctctgcattctctgcttttaaaaaaaccattgcaggtgccagtgtcccatatgttcctcctgacagtttgatgtgtccattctgggcctctcagtgcttagcaagtagataatgtaagggatgtggcagcaaatggaaatgactacaaacactctcctatcaatcacttcaggctacttttatgagttagccagatgcttgtgtatcctcagaccaaactgattcatgtacaaataataaaatgtttactcttttgtaagattatgttttacttatctcaaaggagatacatataatttataatgatatgggcagttgcttccagggacatcaacaaagctgcttagatataatattagataaatataacagaccactctgtattaatggattaaagccagctagttaaacaaccctttttaaccataatcatggaagctttattcttgcaataaagatttttaggctgggcgcagtgactcacacctgtaatcccagcactttgggaagctaaggcaggcagatcatttgaggtcaggagtttgagaccagcctggccaacatggtgaaaccccatctctgctaaaattacaaaaaagttagccgggcatggtggtgtgcacctgtaatcccagctactcgggaggctgaggcaggagaatcacttgaacccgggaggcagaggttgcagtgagccgagatcatgtcactgcactctagcttgggagacagagcgagactccgtctcaaaaaacaaacaaacaaataaaaacacccatttttaacaaaacaactttatatagcatacagccatgattctaaatagtatgattatggttctcaggatctgactacataggtaaaaatatttgcatatgtgtatgaagtgttgggggatgtaggctagaattgtagtctgtgttctaattttggttctaccaccaattagctgtatgacctttagcaagtcctttaacttttcttagattccagggactcatttataaaatgacatggacaaaagcatctctaatcactctaaaagatttgaagtctaggacctaaattctaaatactcttttgaggagtgactgagttttcattttcataattatgtctctcagaggacaaatttacattttcttaacagagacattttcttcttctttttttttgtttgagacagagtctcgctctgtcgtccaggctggagtgcagtgctgcaatcttggctcactgcaacctgcgcctcctgggttcaagtgattcttctgcctcaacctcccaagtagctagacctataggcgcctgccaccatgcccagctaatttttgtatttttagtagagacagggtttcatattggccagactggtctcgaactcctgaccttgtgatccgcccacctcggcctcccaaagtgctgggattacaggtgtgagccaccacacccagccaacattttcctcttttaaaaaatatcttctcacgcctgtaatcccagcactttgggaggctgaggcaggcggatcatgaggtcaggagatcaagaccatcctggctaacacggtgaaactccatctctactaaaaatacaaaaaaaatagccgggcgtggtggcaggcgcctgtagtcccagctactggggaggctgaggcaggaaaatggtgtcaacccgggaggcggagcttgcagtgagccgagattgcgccactgcactccagcctgggcaatagagtgagactccgtctcaaaaaaaaaaaaaaaaaaaaaaacttcaacaataccctcaggttgataattttggatatctatctgtatctatatatcttgtttacctggtctccagaaaaagaacacatacacatatccatatataaaatatgtatacatgtatcaaatctacgtaaactataaaggtgggatggctttaattatggcccaagctactaagacaatgaagactttttggggctgcaagctactgcttcccttctttatctactagcctcttaaacaaggctcacttgtgctacaagacagtccaccgttttgttttttttttcttttttttgagacagggtctcactctttcccaggctgcagtacagtgacacagtctcagctcactgcagctttgaccttgccgggctcaggtgacccttacacttcagcctcccaagtagcagggactataggtgtgcaccaacatgcttggttaatttttgtattttttgtagagacagggttttgccatgttgtccaggctagtctcgaattcctgggctcaagtgattcacctgccttggcctcccaaagtgctaggattacagatgggagccaccacgcccagcccagtccagctcttatatgtagcacagggaaaggacaaatacttgtcaactataaataagaaacattgctaatgcattgcaaagaacactagtttcatttactttataacttagatgtctactgggtgagacgaatgtctttgttctttaaaaaataggaaaagagaagaaaaactagcataacataagtactcatttgtaagactttctgacatgtaacattagttccgtagttttgagacctggtagaactgactttcatatttggataacctggaaaacacccaaacacaaacttcaagtcttctttctcttttttcattatcttttttagtctgaggtgacaccatcattaaggattcgacacccgtttgtaaataaaatgacatcagcaattactctgaaatgtttctagtttgcaaagacttagcaatgtgatgttattaacccttcctcccttcagagacctgtcctaagctctgaaccactcattccttccactcttcttaccccaggtggttgatgagcagtggtccctggtgttccacaaagagtcattaaagtgttacagctggtagcactggtagcaaaaaaacaaaccaaaaagtacacacagacacacacacacacacgcacacatacacacacacacgcacttggccaagtgacaaaagcttggcccctgaaatttctatgagatccgatgaccaccaacatcaaagcatttttttttttttttttttgagacgtagtctcgctctgtcacccaggctagagtgcagtggtgcaatcacagctcactgcaacctccacctcccgggttcaagcgattctcctgcctcagcctctcgagtagctgtgactacaggcacctgccaccatgcccggctaattttttgtatttttagtagagacggggtttcaccgtgttagccaggatggtcttgatctcctgacctcgtgatccatccgcctcggcctcccaaattgctgggattacaggcatgagccaccacgcccggcccatcaaaggaattgtaacaactatttgagagcactgacaataagattaacactcggttgatttagatgttatgctggtcctcaggcattcatctttagatatttttggggtggaagtggggtagggctgacttagtaaaaataacctcttagcccaaaggctttattcagacttacaccgatttgaggggtgggtttgtggaatgcaaggttaggttcttacctaatatttgatgactaatttagaattttaaatgtaattttaaattttagtgactggtttcaaatctattttaacttctagattgttcaaagaggtctcagtacatggctacaatcaaagtattagactagctatttctcagctcagtgctcagaaaaattattactgttgatacctttttctttgtttcctgttaaataaatcacctctttaaagacagaaaaaaaaaaaaaa

SEQ ID NO: 13 - DNA, 909 bp, Homo sapiens
ggctgcttcttttggagggagccagtgggggacgggagtctcagcaggctggggtgttgcacttttccttttgtctgcagcctgatgtttgctttgtgctgaaggaagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctctgcccacccttcaggtggttcccttgtgacaccgttcatccccagatcactgaggccaggccatgtttggggccttgttctgacagcattctggctgaggctggtcggtagcactcctggctggtttttttctgttcctccccgagaggccctctggcccccaggaaacctgttgtgcagagctcttccccggagacctccacacaccctggctttgaagtggagtctgtgactgctctgcattctctgcttttaaaaaaaccattgcaggtgccagtgtcccatatgttcctcctgacagtttgatgtgtccattctgggcctctcagtgcttagcaagtagataatgtaagggatgtggcagcaaatggaaatgactacaaacactctcctatcaatcacttcaggctacttttatgagttagccagatgcttgtgtatcctcagaccaaactgattcatgtacaaataataaaatgtttactcttttgtaa

SEQ ID NO: 14 - DNA, 4523 bp, Homo sapiens
ataggaaagaaggaaatgaaggcggagagtggggagagagcagcagaattcaatttaatctgaggccaccccaagtgttttgtagaattgggctgaagaggtttcctaggcagcctcgcaggctgcacagcacaccacccccgagaagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctctgcccacccttcaggtggttcccttgtgacaccgttcatccccagatcactgaggccaggccatgtttggggccttgttctgacagcattctggctgaggctggtcggtagcactcctggctggtttttttctgttcctccccgagaggccctctggcccccaggaaacctgttgtgcagagctcttccccggagacctccacacaccctggctttgaagtggagtctgtgactgctctgcattctctgcttttaaaaaaaccattgcaggtgccagtgtcccatatgttcctcctgacagtttgatgtgtccattctgggcctctcagtgcttagcaagtagataatgtaagggatgtggcagcaaatggaaatgactacaaacactctcctatcaatcacttcaggctacttttatgagttagccagatgcttgtgtatcctcagaccaaactgattcatgtacaaataataaaatgtttactcttttgtaagattatgttttacttatctcaaaggagatacatataatttataatgatatgggcagttgcttccagggacatcaacaaagctgcttagatataatattagataaatataacagaccactctgtattaatggattaaagccagctagttaaacaaccctttttaaccataatcatggaagctttattcttgcaataaagatttttaggctgggcgcagtgactcacacctgtaatcccagcactttgggaagctaaggcaggcagatcatttgaggtcaggagtttgagaccagcctggccaacatggtgaaaccccatctctgctaaaattacaaaaaagttagccgggcatggtggtgtgcacctgtaatcccagctactcgggaggctgaggcaggagaatcacttgaacccgggaggcagaggttgcagtgagccgagatcatgtcactgcactctagcttgggagacagagcgagactccgtctcaaaaaacaaacaaacaaataaaaacacccatttttaacaaaacaactttatatagcatacagccatgattctaaatagtatgattatggttctcaggatctgactacataggtaaaaatatttgcatatgtgtatgaagtgttgggggatgtaggctagaattgtagtctgtgttctaattttggttctaccaccaattagctgtatgacctttagcaagtcctttaacttttcttagattccagggactcatttataaaatgacatggacaaaagcatctctaatcactctaaaagatttgaagtctaggacctaaattctaaatactcttttgaggagtgactgagttttcattttcataattatgtctctcagaggacaaatttacattttcttaacagagacattttcttcttctttttttttgtttgagacagagtctcgctctgtcgtccaggctggagtgcagtgctgcaatcttggctcactgcaacctgcgcctcctgggttcaagtgattcttctgcctcaacctcccaagtagctagacctataggcgcctgccaccatgcccagctaatttttgtatttttagtagagacagggtttcatattggccagactggtctcgaactcctgaccttgtgatccgcccacctcggcctcccaaagtgctgggattacaggtgtgagccaccacacccagccaacattttcctcttttaaaaaatatcttctcacgcctgtaatcccagcactttgggaggctgaggcaggcggatcatgaggtcaggagatcaagaccatcctggctaacacggtgaaactccatctctactaaaaatacaaaaaaaatagccgggcgtggtggcaggcgcctgtagtcccagctactggggaggctgaggcaggaaaatggtgtcaacccgggaggcggagcttgcagtgagccgagattgcgccactgcactccagcctgggcaatagagtgagactccgtctcaaaaaaaaaaaaaaaaaaaaaaacttcaacaataccctcaggttgataattttggatatctatctgtatctatatatcttgtttacctggtctccagaaaaagaacacatacacatatccatatataaaatatgtatacatgtatcaaatctacgtaaactataaaggtgggatggctttaattatggcccaagctactaagacaatgaagactttttggggctgcaagctactgcttcccttctttatctactagcctcttaaacaaggctcacttgtgctacaagacagtccaccgttttgttttttttttcttttttttgagacagggtctcactctttcccaggctgcagtacagtgacacagtctcagctcactgcagctttgaccttgccgggctcaggtgacccttacacttcagcctcccaagtagcagggactataggtgtgcaccaacatgcttggttaatttttgtattttttgtagagacagggttttgccatgttgtccaggctagtctcgaattcctgggctcaagtgattcacctgccttggcctcccaaagtgctaggattacagatgggagccaccacgcccagcccagtccagctcttatatgtagcacagggaaaggacaaatacttgtcaactataaataagaaacattgctaatgcattgcaaagaacactagtttcatttactttataacttagatgtctactgggtgagacgaatgtctttgttctttaaaaaataggaaaagagaagaaaaactagcataacataagtactcatttgtaagactttctgacatgtaacattagttccgtagttttgagacctggtagaactgactttcatatttggataacctggaaaacacccaaacacaaacttcaagtcttctttctcttttttcattatcttttttagtctgaggtgacaccatcattaaggattcgacacccgtttgtaaataaaatgacatcagcaattactctgaaatgtttctagtttgcaaagacttagcaatgtgatgttattaacccttcctcccttcagagacctgtcctaagctctgaaccactcattccttccactcttcttaccccaggtggttgatgagcagtggtccctggtgttccacaaagagtcattaaagtgttacagctggtagcactggtagcaaaaaaacaaaccaaaaagtacacacagacacacacacacacacgcacacatacacacacacacgcacttggccaagtgacaaaagcttggcccctgaaatttctatgagatccgatgaccaccaacatcaaagcatttttttttttttttttttgagacgtagtctcgctctgtcacccaggctagagtgcagtggtgcaatcacagctcactgcaacctccacctcccgggttcaagcgattctcctgcctcagcctctcgagtagctgtgactacaggcacctgccaccatgcccggctaattttttgtatttttagtagagacggggtttcaccgtgttagccaggatggtcttgatctcctgacctcgtgatccatccgcctcggcctcccaaattgctgggattacaggcatgagccaccacgcccggcccatcaaaggaattgtaacaactatttgagagcactgacaataagattaacactcggttgatttagatgttatgctggtcctcaggcattcatctttagatatttttggggtggaagtggggtagggctgacttagtaaaaataacctcttagcccaaaggctttattcagacttacaccgatttgaggggtgggtttgtggaatgcaaggttaggttcttacctaatatttgatgactaatttagaattttaaatgtaattttaaattttagtgactggtttcaaatctattttaacttctagattgttcaaagaggtctcagtacatggctacaatcaaagtattagactagctatttctcagctcagtgctcagaaaaattattactgttgatacctttttctttgtttcctgttaaataaatcacctctttaaagacagaaaaaaaaaaaaaa